SEQUENCE LISTING (122 sequences)

SEQ ID NO: 1 - DNA, 23 bp, Artificial sequence
  synthetic polynucleotide
  misc_feature: EV71_3D guide1 sequence
gttggtccattgatgtttagtct

SEQ ID NO: 2 - DNA, 23 bp, Artificial sequence
  Synthetic polynucleotide
  misc_feature: EV71_3D guide2 sequence
ttggaaaacagggcttgttcaaa

SEQ ID NO: 3 - DNA, 23 bp, Artificial sequence
  Synthetic polynucleotide
  misc_feature: EV71_3D guide3 sequence
tagcacgcttcctccatgctcat

SEQ ID NO: 4 - DNA, 23 bp, Artificial sequence
  Synthetic polynucleotide
  misc_feature: EV71_3D guide4 sequence
tatttatccatgtagaatttcat

SEQ ID NO: 5 - DNA, 23 bp, Artificial sequence
  Synthetic polynucleotide
  misc_feature: EV71_3D guide5 sequence
atgatgttgttgatcattgaatt

SEQ ID NO: 6 - DNA, 29 bp, Artificial sequence
  Synthetic polynucleotide
  misc_feature: EV71_3D guide6 sequence
tctgcaggagtcatggtcaaaccatactc

SEQ ID NO: 7 - DNA, 23 bp, Artificial sequence
  Synthetic polynucleotide
  misc_feature: GFP guide1 sequence
gtgaacagctcctcgcccttgct

SEQ ID NO: 8 - DNA, 21 bp, Artificial sequence
  Synthetic polynucleotide
  misc_feature: GFP guide2 sequence
gctgaacttgtggccgtttac

SEQ ID NO: 9 - DNA, 35 bp, Artificial sequence
  Synthetic polynucleotide
  misc_feature: GG001; Primer for cloning pZac-CMV-CasRx- polyA-U6-gRNA
atggtctccatctgcttatatagacctcccaccgt

SEQ ID NO: 10 - DNA, 31 bp, Artificial sequence
  Synthetic polynucleotide
  misc_feature: GG002; Primer for cloning pZac-CMV-CasRx- polyA-U6-gRNA
atggtctcaagatgggcgtgaagtccacact

SEQ ID NO: 11 - DNA, 30 bp, Artificial sequence
  Synthetic polynucleotide
  misc_feature: GG003; Primer for cloning pZac-CMV-CasRx- polyA-U6-gRNA
atggtctcctatccggatccggaattgccg

SEQ ID NO: 12 - DNA, 38 bp, Artificial sequence
  Synthetic polynucleotide
  misc_feature: GG004; Primer for cloning pZac-CMV-CasRx- polyA-U6-gRNA
atggtctcggatacccatacgatgttccagattacgct

SEQ ID NO: 13 - DNA, 37 bp, Artificial sequence
  Synthetic polynucleotide
  misc_feature: GG005; Primer for cloning pZac-CMV-CasRx- polyA-U6-gRNA
taggtctcacattaggcatagtcggggacatcatatg

SEQ ID NO: 14 - DNA, 50 bp, Artificial sequence
  Synthetic polynucleotide
  misc_feature: GG006; Primer for cloning pZac-CMV-CasRx- polyA-U6-gRNA
atggtctccaatggctaataaaggaaatttattttcattgcaatagtgtg

SEQ ID NO: 15 - DNA, 39 bp, Artificial sequence
  Synthetic polynucleotide
  misc_feature: GG007; Primer for cloning pZac-CMV-CasRx- polyA-U6-gRNA
atggtctcactcgatctccataagagaagagggacagct

SEQ ID NO: 16 - DNA, 36 bp, Artificial sequence
  Synthetic polynucleotide
  misc_feature: GG008; Primer for cloning pZac-CMV-CasRx- polyA-U6-gRNA
atggtctcccgagggcctatttcccatgattccttc

SEQ ID NO: 17 - DNA, 35 bp, Artificial sequence
  Synthetic polynucleotide
  misc_feature: GG009; Primer for cloning pZac-CMV-CasRx- polyA-U6-gRNA
atggtctcaattcacgacacctgaaatggaagaaa

SEQ ID NO: 18 - DNA, 31 bp, Artificial sequence
  Synthetic polynucleotide
  misc_feature: GG0010; Primer for cloning pZac-CMV-CasRx- polyA-U6-gRNA
atggtctcggaatgggcgtgaagtccacact

SEQ ID NO: 19 - DNA, 45 bp, Artificial sequence
  Synthetic polynucleotide
  misc_feature: EK19001; Primer for cloning EV71_3D_guide1 into pZac backbone
cctaccaactggtcggggtttggttggtccattgatgtttagtct

SEQ ID NO: 20 - DNA, 50 bp, Artificial sequence
  Synthetic polynucleotide
  misc_feature: EK19002; Primer for cloning EV71_3D_guide1 into pZac backbone
ggagaaaataccgcatcagaattcaaaagactaaacatcaatggaccaac

SEQ ID NO: 21 - DNA, 45 bp, Artificial sequence
  Synthetic polynucleotide
  misc_feature: EK19003; Primer for cloning EV71_3D_guide2 into pZac backbone
cctaccaactggtcggggtttgttggaaaacagggcttgttcaaa

SEQ ID NO: 22 - DNA, 50 bp, Artificial sequence
  Synthetic polynucleotide
  misc_feature: EK19004; Primer for cloning EV71_3D_guide2 into pZac backbone
ggagaaaataccgcatcagaattcaaatttgaacaagccctgttttccaa

SEQ ID NO: 23 - DNA, 45 bp, Artificial sequence
  Synthetic polynucleotide
  misc_feature: EK19005; Primer for cloning EV71_3D_guide3 into pZac backbone
cctaccaactggtcggggtttgtagcacgcttcctccatgctcat

SEQ ID NO: 24 - DNA, 50 bp, Artificial sequence
  Synthetic polynucleotide
  misc_feature: EK19006; Primer for cloning EV71_3D_guide3 into pZac backbone
ggagaaaataccgcatcagaattcaaaatgagcatggaggaagcgtgcta

SEQ ID NO: 25 - DNA, 45 bp, Artificial sequence
  Synthetic polynucleotide
  misc_feature: EK19007; Primer for cloning EV71_3D_guide4 into pZac backbone
cctaccaactggtcggggtttgtatttatccatgtagaatttcat

SEQ ID NO: 26 - DNA, 50 bp, Artificial sequence
  Synthetic polynucleotide
  misc_feature: EK19008; Primer for cloning EV71_3D_guide4 into pZac backbone
ggagaaaataccgcatcagaattcaaaatgaaattctacatggataaata

SEQ ID NO: 27 - DNA, 45 bp, Artificial sequence
  Synthetic polynucleotide
  misc_feature: EK19009; Primer for cloning EV71_3D_guide5 into pZac backbone
cctaccaactggtcggggtttgatgatgttgttgatcattgaatt

SEQ ID NO: 28 - DNA, 50 bp, Artificial sequence
  Synthetic polynucleotide
  misc_feature: EK19010; Primer for cloning EV71_3D_guide5 into pZac backbone
ggagaaaataccgcatcagaattcaaaaattcaatgatcaacaacatcat

SEQ ID NO: 29 - DNA, 51 bp, Artificial sequence
  Synthetic polynucleotide
  misc_feature: EK19011; Primer for cloning EV71_3D_guide6 into pZac backbone
cctaccaactggtcggggtttgtctgcaggagtcatggtcaaaccatactc

SEQ ID NO: 30 - DNA, 56 bp, Artificial sequence
  Synthetic polynucleotide
  misc_feature: EK19012; Primer for cloning EV71_3D_guide6 into pZac backbone
ggagaaaataccgcatcagaattcaaagagtatggtttgaccatgactcctgcaga

SEQ ID NO: 31 - DNA, 33 bp, Artificial sequence
  Synthetic polynucleotide
  misc_feature: SV038; primer for cloning GFP_guide1 into pZac backbone
aactggtcggggtttggtgaacagctcctcgcc

SEQ ID NO: 32 - DNA, 44 bp, Artificial sequence
  Synthetic polynucleotide
  misc_feature: SV039; primer for cloning GFP_guide1 into pZac backbone
gaaaataccgcatcagaattcaaaaaaaagcaagggcgaggagc

SEQ ID NO: 33 - DNA, 34 bp, Artificial sequence
  Synthetic polynucleotide
  misc_feature: SV040; primer for cloning GFP_guide2 into pZac backbone
aactggtcggggtttggctgaacttgtggccgtt

SEQ ID NO: 34 - DNA, 41 bp, Artificial sequence
  Synthetic polynucleotide
  misc_feature: SV041; primer for cloning GFP_guide2 into pZac backbone
ccgcatcagaattcaaaaaaagtaaacggccacaagttcag

SEQ ID NO: 35 - DNA, 1395 bp, Artificial sequence
  Synthetic polynucleotide
  misc_feature: 3D_protein_guides_desig 1395 bp ds-DNA
  misc_feature (1)..(1392): CDS
  misc_feature (1)..(1392): mature peptide Geneious name 3D peptide
  misc_feature (46)..(68): Guide1
  misc_feature (166)..(188): Guide2
  misc_feature (289)..(311): Guide3
  misc_feature (427)..(449): Guide4
  misc_feature (898)..(920): Guide5
  misc_feature (1054)..(1082): Guide6
atgcaaggtgagatccaatgggtgaagcctaacaaggaaactggcagactaaacatcaatggaccaactcgcactaagttggagcctagtgtatttcatgatgtgtttgaaggcaacaaggaaccagcagttttaacaagtaaagaccctagattggaggtcgactttgaacaagccctgttttccaagtatgtgggcaatgttttacacgagcccgatgaatatgtgactcaagctgccctccactatgcgaatcaacttaaacaattggacataaacactagcaagatgagcatggaggaagcgtgctatggcactgaaaacctggaagcaatagacctctgcactagtgctgggtatccatacagtgcccttggtatcaagaaaagagacattctcgaccccataaccagggatgtgtctaagatgaaattctacatggataaatacggactagatctgccatactctacctatgtgaaggatgaacttagatctctggataaaatcaagaaaggaaagtcacgcctgatagaggccagcagcttgaatgactctgtctacctcagaatgacttttgggcacctttacgaggtgtttcatgctaaccctggtactgtgactggctcagcagtaggttgcaacccagacgtgttttggagtaaactaccgattctgctgcctgggtcactctttgcctttgactactcaggatatgatgctagtctcagcccggtatggttcagggctctagaagttgtgttacgggagattgggtattcagaggaggccgtgtccctaatagaaggaatcaaccacacccaccatgtgtaccggaataaaacatactgtgtacttggtgggatgccctcagggtgctctggtacttccatcttcaattcaatgatcaacaacatcatcattagaacccttttgatcaaaacctttaagggaatagacctggatgagttgaacatggtggcctatggggacgatgtgctggccagttacccttttcctattgattgccttgaattggctaagactggcaaagagtatggtttgaccatgactcctgcagacaaatcaccctgtttcaatgaagtaacatgggagaatgctaccttcctgaagagagggttcttgccagaccaccaatttccattcttaattcaccctacgatgcccatgagagagatccatgagtccattcgatggactaaggacgcgcgtaacacccaggatcacgtgcgctccctgtgtctattggcatggcacaatggtaaggatgaatatgaaaagtttgtgagtgcaattagatcagttccagttggaaaagcgttggccattcctaactttgagaatctgagaagaaattggctcgaattgttttaa

SEQ ID NO: 36 - DNA, 746 bp, Artificial sequence
  Synthetic polynucleotide
  misc_feature: GFP_guides_design 746 bp ds-DNA circular
  misc_feature (1)..(746): CDS - eGFP
  misc_feature (7)..(29): Knockdown_Guide1
  misc_feature (67)..(87): Knockdown_Guide2
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatcacgacgacgagctgtacaagtaa

SEQ ID NO: 37 - DNA, 7508 bp, Artificial sequence
  Synthetic polynucleotide
  misc_feature: pZac2.1-CMV-CasRx-3xHA- 7508 bp ds-DNA circular
  misc_feature (121)..(144): primer complement(121..144) /label=“2225” /note=“sequence GGGAGAGGCGGTTTGCGTA TTGGG” (SEQ ID NO 38)
  repeat_region (364)..(493): ITR
  misc_feature (534)..(557): primer 534..557/label=“EK18011” /note=“sequence gccatgctctaggaagatcgggac”(SEQ ID NO 39)
  misc_feature (534)..(593): primer 534..593/label=“EK18001”/note=“sequence gccatgctctaggaagatcgggacattgattattgactagtta ttaatagtaatcaatta” (SEQ ID NO 40)
  enhancer (555)..(934): CMV enhancer
  misc_feature (555)..(934): CDS
  misc_feature (622)..(645): primer 622..645/label=“1075” /note=“sequence CGGAGTTCCGCGTTACATAACTTAC” (SEQ ID NO 41)
  misc_feature (722)..(741): primer 722..741/label=“1324” /note=“sequence ACGCCAATAGGGACTTTCCA” (SEQ ID NO 42)
  misc_feature (811)..(830): primer 811..830/label=“2394” /note=“sequence AAGTACGCCCCCTATTGACG” (SEQ ID NO 43)
  promoter (936)..(1134): CMV promoter
  misc_feature (1052)..(1077): primer complement(1052..1077)/label=“EK18015” /note=“sequence gagttgttacgacattttgg aaagtc” (SEQ ID NO 44)
  misc_feature (1111)..(1134): primer complement(1111..1134) /label=“GG001” /note=“sequence ATGGTCTCCatctgcttatatag acctcccaccgt” (SEQ ID NO 45)
  misc_feature (1111)..(1134): primer complement(1111..1134)/label=“SV036” /note=“sequence tgtggacttcacgcccatggtggcctgc ttatatagacctcccaccgt” (SEQ ID NO 46)
  misc_feature (1135)..(1140): Kozak
  misc_feature (1141)..(4017): CasRx
  misc_feature (1141)..(4017): CDS Translation 1141-4017
  misc_feature (4018)..(4044): primer 4018..4044 /label=“GG004” /note=“sequence ATGGTCTCGgatacccatacgatgttccaga ttacgct” (SEQ ID NO 47)
  misc_feature (4018)..(4098): CDS - 3xHA
  misc_feature (4076)..(4100): primer complement(4076..4100) /label=“GG005” /note=“sequence TAGGTCTCAcattaggcatagt cggggacatcatatg”(SEQ ID NO 48)
  misc_feature (4102)..(4140): primer 4102..4140 /label=“GG006” /note=“sequence ATGGTCTCCaatggctaataaaggaaatttattttca ttgcaatagtgtg” (SEQ ID NO 49)
  polyA_signal (4102)..(4488): rabbit b-globin polyA
  misc_feature (4462)..(4488): primer complement(4462..4488) /label=“GG007” /note=“sequence ATGGTCTCActcgatctccata agagaagagggacagct” (SEQ ID NO 50)
  misc_feature (4489)..(4510): primer 4489..4510 /label=“U6_fwd_seqchk” /note=“sequence gagggcctatttcccatgattc” (SEQ ID NO 51)
  misc_feature (4489)..(4514): primer 4489..4514 /label=“GG008” /note=“sequence ATGGTCTCCcgagggcctatttcccatgatt ccttc”(SEQ ID NO 52)
  promoter (4489)..(4729): U6 promoter
  misc_feature (4490)..(4511): primer complement(4490..4511) /label=“13 EF1a_R (out)” /note=“sequence ggaatcatgggaaata ggccct” (SEQ ID NO 53)
  primer_bind (4660)..(4679): LKO.1 5′
  misc_feature (4713)..(4738): primer complement(4713..4738) /label=“736” /note=“sequence CGGTGTTTCGTCCTTTCCA CAAGATA” (SEQ ID NO 54)
  misc_feature (4743)..(4759): primer 4743..4759 /label=“EK_GS_F1” /note=“sequence cctaccaactggtcggg”(SEQ ID NO 55)
  misc_feature (4743)..(4764): primer 4743..4764 /label=“EK18100” /note=“sequence cctaccaactggtcggggtttg” (SEQ ID NO 56)
  misc_feature (4791)..(4817): primer complement(4791..4817) /label=“EK18101” /note=“sequence GGAGAAAATACCGCA TCAGAATTCAAA” (SEQ ID NO 57)
  misc_feature (4796)..(4817): primer complement(4796..4817) /label=“EK_GS_R1” /note=“sequence ggagaaaataccgcatcag aat” (SEQ ID NO 58)
  misc_feature (4843)..(4895): primer complement(4843..4895) /label=“EK18008” /note=“sequence gctacttatctacgtagccatgcg aattactatggttgctttgacgtatgcgg” (SEQ ID NO 59)
  misc_feature (4866)..(4895): primer complement(4866..4895) /label=“EK18012” /note=“sequence gctacttatctacgtagccatg cgaattac” (SEQ ID NO 60)
  misc_feature (4868)..(7508): join(4868..7508,1..554) /label=“pZac2.1 BB
  misc_feature (4920)..(5060): ITR
  misc_feature (5206)..(5225): primer complement(5206..5225) /label=”2452“ /note=”sequence TTCAGGCTGCGCAACTGT TG“ (SEQ ID NO 61)
  misc_feature (5824)..(5849): primer 5824..5849 /label=”1625“ /note=”sequence ATGAGTATTCAACATTTCCGTGTCGC“ (SEQ ID NO 62)
  misc_feature (7226)..(7246): primer complement(7226..7246) /label=”2372“ /note=”sequence GCTCACGCTGTAGGT ATCTCA“ (SEQ ID NO 63)
tcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcaggccaccatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggatacccatacgatgttccagattacgcttatccctacgacgtgcctgattatgcatacccatatgatgtccccgactatgcctaatggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacgggtcttcgagaagaccttttttttgaattctgatgcggtattttctccttacgcatctgtgcggtatttcacaccgcatacgtcaaagcaaccatagtaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctt

SEQ ID NO: 38 - DNA, 24 bp, Artificial sequence
  Synthetic polynucleotide
  misc_feature: primer complement(121..144) /label=”2225“of SEQ ID NO 37
gggagaggcggtttgcgtattggg

SEQ ID NO: 39 - DNA, 24 bp, Artificial sequence
  Synthetic polynucleotide
  misc_feature: primer 534..557 /label=”EK18011“ of SEQ ID NO 37
gccatgctctaggaagatcgggac

SEQ ID NO: 40 - DNA, 60 bp, Artificial sequence
  Synthetic polynucleotide
  misc_feature: primer 534..593 /label=”EK18001“ of SEQ ID NO 37
gccatgctctaggaagatcgggacattgattattgactagttattaatagtaatcaatta

SEQ ID NO: 41 - DNA, 25 bp, Artificial sequence
  Synthetic polynucleotide
  misc_feature: primer 622..645 /label=”1075“ of SEQ ID NO 37
cggagttccgcgttacataacttac

SEQ ID NO: 42 - DNA, 20 bp, Artificial sequence
  Synthetic polynucleotide
  misc_feature: primer 722..741/label=”1324“ of SEQ ID NO 37
acgccaatagggactttcca

SEQ ID NO: 43 - DNA, 20 bp, Artificial sequence
  Synthetic polynucleotide
  misc_feature: primer 811..830/label=”2394“ of SEQ ID NO 37
aagtacgccccctattgacg

SEQ ID NO: 44 - DNA, 26 bp, Artificial sequence
  Synthetic polynucleotide
  misc_feature: primer complement(1052..1077) /label=”EK18015“ of SEQ ID NO 37
gagttgttacgacattttggaaagtc

SEQ ID NO: 45 - DNA, 35 bp, Artificial sequence; identical to SEQ ID NO: 9
  Synthetic polynucleotide
  misc_feature: primer  complement(1111..1134) /label=”GG001“ of SEQ ID NO 37

SEQ ID NO: 46 - DNA, 48 bp, Artificial sequence
  Synthetic polynucleotide
  misc_feature: primer complement(1111..1134) /label=”SV036“ of SEQ ID NO 37
tgtggacttcacgcccatggtggcctgcttatatagacctcccaccgt

SEQ ID NO: 47 - DNA, 38 bp, Artificial sequence; identical to SEQ ID NO: 12
  Synthetic polynucleotide
  misc_feature: primer 4018..4044/label=”GG004“ of SEQ ID NO 37

SEQ ID NO: 48 - DNA, 37 bp, Artificial sequence; identical to SEQ ID NO: 13
  Synthetic polynucleotide
  misc_feature: primer complement(4076..4100)/label=”GG005“of SEQ ID NO 37

SEQ ID NO: 49 - DNA, 50 bp, Artificial sequence; identical to SEQ ID NO: 14
  Synthetic polynucleotide
  misc_feature: primer 4102..4140 /label=”GG006“ of SEQ ID NO 37

SEQ ID NO: 50 - DNA, 39 bp, Artificial sequence; identical to SEQ ID NO: 15
  Synthetic polynucleotide
  misc_feature: primer complement(4462..4488) /label=”GG007“of SEQ ID NO 37

SEQ ID NO: 51 - DNA, 22 bp, Artificial sequence
  Synthetic polynucleotide
  misc_feature: primer 4489..4510 /label=”U6_fwd_seqchk“ of SEQ ID NO 37
gagggcctatttcccatgattc

SEQ ID NO: 52 - DNA, 36 bp, Artificial sequence; identical to SEQ ID NO: 16
  Synthetic polynucleotide
  misc_feature: primer 4489..4514 /label=”GG008“ of SEQ ID NO 37

SEQ ID NO: 53 - DNA, 22 bp, Artificial sequence
  Synthetic polynucleotide
  misc_feature: Primer complement(4490..4511) /label=”13 EF1a_R (out)“ of SEQ ID NO 37
ggaatcatgggaaataggccct

SEQ ID NO: 54 - DNA, 26 bp, Artificial sequence
  Synthetic polynucleotide
  misc_feature: primer complement(4713..4738) /label=”736“ of SEQ ID NO 37
cggtgtttcgtcctttccacaagata

SEQ ID NO: 55 - DNA, 17 bp, Artificial sequence
  Synthetic polynucleotide
  misc_feature: primer 4743..4759 /label=”EK_GS_F1“ of SEQ ID NO 37
cctaccaactggtcggg

SEQ ID NO: 56 - DNA, 22 bp, Artificial sequence
  Synthetic polynucleotide
  misc_feature: primer 4743..4764 /label=”EK18100“ of SEQ ID NO 37
cctaccaactggtcggggtttg

SEQ ID NO: 57 - DNA, 27 bp, Artificial sequence
  Synthetic polynucleotide
  misc_feature: primer complement(4791..4817) /label=”EK18101“ of SEQ ID NO 37
ggagaaaataccgcatcagaattcaaa

SEQ ID NO: 58 - DNA, 22 bp, Artificial sequence
  Synthetic polynucleotide
  misc_feature: primer complement(4796..4817) /label=”EK_GS_R1“ of SEQ ID NO 37
ggagaaaataccgcatcagaat

SEQ ID NO: 59 - DNA, 53 bp, Artificial sequence
  Synthetic polynucleotide
  misc_feature: primer complement(4843..4895) /label=”EK18008“ of SEQ ID NO 37
gctacttatctacgtagccatgcgaattactatggttgctttgacgtatgcgg

SEQ ID NO: 60 - DNA, 30 bp, Artificial sequence
  Synthetic polynucleotide
  misc_feature: primer complement(4866..4895) /label=”EK18012“ of SEQ ID NO 37
gctacttatctacgtagccatgcgaattac

SEQ ID NO: 61 - DNA, 20 bp, Artificial sequence
  Synthetic polynucleotide
  misc_feature: primer complement(5206..5225) /label=”2452“ of SEQ ID NO 37
ttcaggctgcgcaactgttg

SEQ ID NO: 62 - DNA, 26 bp, Artificial sequence
  Synthetic polynucleotide
  misc_feature: primer 5824..5849 /label=”1625“ of SEQ ID NO 37
atgagtattcaacatttccgtgtcgc

SEQ ID NO: 63 - DNA, 21 bp, Artificial sequence
  Synthetic polynucleotide
  misc_feature: primer complement(7226..7246) /label=”2372“ of SEQ ID NO 37
gctcacgctgtaggtatctca

SEQ ID NO: 64 - DNA, 23 bp, Artificial sequence
  Synthetic polynucleotide
  misc_feature: consensus sequence - untitled consensus; and aligned sequences - EV71-SHENZEN001-2006,and EV71-NJ2017iso2 of FIG. 10
agactcaacatcaatggaccaac

SEQ ID NO: 65 - DNA, 23 bp, Artificial sequence
  Synthetic polynucleotide
  misc_feature: aligned sequence - Strain H8-1 of FIG. 10
agactcaacatcaatgggccaac

SEQ ID NO: 66 - DNA, 23 bp, Artificial sequence
  Synthetic polynucleotide
  misc_feature: aligned sequences - HEVA71-Strain41, Enterovirus-sin002209, and nucleic acid sequence of Guide1 of FIG. 10
agactaaacatcaatggaccaac

SEQ ID NO: 67 - DNA, 23 bp, Artificial sequence
  Synthetic polynucleotide
  misc_feature: aligned sequence - EV71 MZ of FIG. 10
agactcagcatcaatggaccaac

SEQ ID NO: 68 - protein, 8 aa, Artificial sequence
  Synthetic peptide
  MISC_FEATURE: Guide1 protein sequence of FIG. 10
RLNINGPT

SEQ ID NO: 69 - DNA, 23 bp, Artificial sequence
  Synthetic polynucleotide
  misc_feature: consensus sequence - untitled consensus of FIG. 10
tttgaacaagccctgttctccaa

SEQ ID NO: 70 - DNA, 23 bp, Artificial sequence
  Synthetic polynucleotide
  misc_feature: aligned sequence - Strain H8-1 of FIG. 10
tttgaacaggccctgttctccaa

SEQ ID NO: 71 - DNA, 23 bp, Artificial sequence
  Synthetic polynucleotide
  misc_feature: aligned sequence - EV71-SHENZEN001-2006 of FIG. 10
tttgaacaagccctgttctctaa

SEQ ID NO: 72 - DNA, 23 bp, Artificial sequence
  Synthetic polynucleotide
  misc_feature: aligned sequences - HEVA71-Strain41, Enterovirus-sin002209, and nucleic acid sequence of Guide2 of FIG. 10
tttgaacaagccctgttttccaa

SEQ ID NO: 73 - DNA, 23 bp, Artificial sequence
  Synthetic polynucleotide
  misc_feature: aligned sequences - EV71 MZ, and EV71-NJ2017iso2 of FIG. 10
tttgaacaggccctgttctctaa

SEQ ID NO: 74 - protein, 8 aa, Artificial sequence
  Synthetic peptide
  MISC_FEATURE: Guide2 protein sequence of FIG. 10
FEQALFSK

SEQ ID NO: 75 - DNA, 23 bp, Artificial sequence
  Synthetic polynucleotide
  misc_feature: consensus sequence - untitled consensus; and aligned sequences -Strain H8-1, EV71-SHENZEN001-2006, EV71-MZ, and EV71-NJ2017iso2 of FIG. 10
atgagcatggaggaggcctgcta

SEQ ID NO: 76 - DNA, 23 bp, Artificial sequence
  Synthetic polynucleotide
  misc_feature: aligned sequences - HEVA71-Strain41, Enterovirus-sin002209, and nucleic acid sequence of Guide3 of FIG. 10
atgagcatggaggaagcgtgcta

SEQ ID NO: 77 - protein, 8 aa, Artificial sequence
  Synthetic peptide
  MISC_FEATURE: Guide3 protein sequence of FIG. 10
MSMEEACY

SEQ ID NO: 78 - DNA, 23 bp, Artificial sequence
  Synthetic polynucleotide
  misc_feature: consensus sequence - untitled consensus; and aligned sequences -Strain H8-1,and EV71-SHENZEN001-2006 of FIG. 10
atgaagttctacatggacaaata

SEQ ID NO: 79 - DNA, 23 bp, Artificial sequence
  Synthetic polynucleotide
  misc_feature: aligned sequences - HEVA71-Strain41, Enterovirus-sin002209, and nucleic acid sequence of Guide4 of FIG. 10
atgaaattctacatggataaata

SEQ ID NO: 80 - DNA, 23 bp, Artificial sequence
  Synthetic polynucleotide
  misc_feature: aligned sequences - EV71-MZ and EV71-NJ2017iso2 of FIG. 10
atgaagttttacatggacaagta

SEQ ID NO: 81 - protein, 8 aa, Artificial sequence
  Synthetic peptide
  MISC_FEATURE: Guide4 protein sequence of FIG. 10
MKFYMDKY

SEQ ID NO: 82 - DNA, 23 bp, Artificial sequence
  Synthetic polynucleotide
  misc_feature: consensus sequence - untitled consensus; and aligned sequences - Strain H8-1 and EV71-SHENZEN001-2006 of FIG. 10
aactcaatgatcaacaacattat

SEQ ID NO: 83 - DNA, 23 bp, Artificial sequence
  Synthetic polynucleotide
  misc_feature: aligned sequence - HEVA71-Strain41 and nucleic acid sequence of Guide5 of FIG. 10
aattcaatgatcaacaacatcat

SEQ ID NO: 84 - DNA, 23 bp, Artificial sequence
  Synthetic polynucleotide
  misc_feature: aligned sequence - EV71-MZ of FIG. 10.
aactcaatgatcaataacattat

SEQ ID NO: 85 - DNA, 23 bp, Artificial sequence
  Synthetic polynucleotide
  misc_feature: aligned sequence - Enterovirus-sin002209 of FIG. 10
aattcaatgatcaataacatcat

SEQ ID NO: 86 - DNA, 23 bp, Artificial sequence
  Synthetic polynucleotide
  misc_feature: aligned sequence - EV71-NJ2017iso2 of FIG. 10
aattcaatgatcaacaacattat

SEQ ID NO: 87 - protein, 8 aa, Artificial sequence
  Synthetic peptide
  MISC_FEATURE: Guide5 protein sequence of FIG. 10
NSMINNII

SEQ ID NO: 88 - DNA, 29 bp, Artificial sequence
  Synthetic polynucleotide
  misc_feature: consensus sequence - untitled consensus; and aligned sequences - Strain H8-1 and EV71-SHENZEN001-2006 of FIG. 10
gagtatggtctgaccatgactcctgcaga

SEQ ID NO: 89 - DNA, 29 bp, Artificial sequence
  Synthetic polynucleotide
  misc_feature: aligned sequence - HEVA71-Strain41, Enterovirus-sin002209, and nucleic acid sequence of Guide6 of FIG. 10
gagtatggtttgaccatgactcctgcaga

SEQ ID NO: 90 - DNA, 29 bp, Artificial sequence
  Synthetic polynucleotide
  misc_feature: aligned sequence - EV71-MZ of FIG. 10.
gagtatggcttgaccatgactcctgctga

SEQ ID NO: 91 - DNA, 29 bp, Artificial sequence
  Synthetic polynucleotide
  misc_feature: aligned sequence - EV71-NJ2017iso2 of FIG. 10
gagtatggtctgaccatgactcctgctga

SEQ ID NO: 92 - protein, 10 aa, Artificial sequence
  Synthetic peptide
EYGLTMTPAD

SEQ ID NO: 93 - DNA, 23 bp, Artificial sequence
  Synthetic polynucleotide
  misc_feature: consensus sequence - untitled consensus of FIG. 11
ctttgagggcaacaaagaaccag

SEQ ID NO: 94 - DNA, 23 bp, Artificial sequence
  Synthetic polynucleotide
  misc_feature: aligned sequence - LC126150 of FIG. 11
ctttgagggcaacaaggaaccag

SEQ ID NO: 95 - DNA, 23 bp, Artificial sequence
  Synthetic polynucleotide
  misc_feature: aligned sequence - CAU05876; Cox_Guide1 of FIG. 11
ctttgaggggaacaaagaaccag

SEQ ID NO: 96 - DNA, 23 bp, Artificial sequence
  Synthetic polynucleotide
  misc_feature: consensus sequence - untitled consensus of FIG. 11
ctccaagtatgtaggaaacacac

SEQ ID NO: 97 - DNA, 23 bp, Artificial sequence
  Synthetic polynucleotide
  misc_feature: aligned sequence - LC126150 of FIG. 11
ctctaagtatgtaggaaacacac

SEQ ID NO: 98 - DNA, 23 bp, Artificial sequence
  Synthetic polynucleotide
  misc_feature: aligned sequence - CAU05876; Cox_Guide2 of FIG. 11
ctccaagtatgtagggaacacac

SEQ ID NO: 99 - DNA, 23 bp, Artificial sequence
  Synthetic polynucleotide
  misc_feature: consensus sequence - untitled consensus of FIG. 11; aligned sequence - CAU05876; Cox_Guide3 of FIG. 11
cactatgcaaatcagttgaagca

SEQ ID NO: 100 - DNA, 23 bp, Artificial sequence
  Synthetic polynucleotide
  misc_feature: aligned sequence - LC126150 of FIG. 11
cattatgcaaatcagttgaagca

SEQ ID NO: 101 - DNA, 23 bp, Artificial sequence
  Synthetic polynucleotide
  misc_feature: consensus sequence - untitled consensus of FIG. 11
gatgtgagcaagatgaaattcta

SEQ ID NO: 102 - DNA, 23 bp, Artificial sequence
  Synthetic polynucleotide
  misc_feature: aligned sequence - LC126150 of FIG. 11
gatgtgagcaagatgaagttcta

SEQ ID NO: 103 - DNA, 23 bp, Artificial sequence
  Synthetic polynucleotide
  misc_feature: aligned sequence - CAU05876; Cox_Guide4 of FIG. 11
gatgtgagtaagatgaaattcta

SEQ ID NO: 104 - DNA, 23 bp, Artificial sequence
  Synthetic polynucleotide
  misc_feature: consensus sequence - untitled consensus; aligned sequences - LC126150 and CAU05876; Cox_Guide5 of FIG. 11
tgccctcaggctgttcaggaaca

SEQ ID NO: 105 - DNA, 23 bp, Artificial sequence
  Synthetic polynucleotide
  misc_feature: consensus sequence - untitled consensus; aligned sequence - CAU05876; Cox_Guide6 of FIG. 11
caactcaatgatcaacaacatca

SEQ ID NO: 106 - DNA, 23 bp, Artificial sequence
  Synthetic polynucleotide
  misc_feature: aligned sequence - LC126150 of FIG. 11
caactcaatgattaacaacatca

SEQ ID NO: 107 - DNA, 24 bp, Artificial sequence
  Synthetic polynucleotide
  misc_feature: consensus sequence - untitled consensus; aligned sequences - AF465516; Echo_guide1 and X89538_Echo7_3D of FIG. 12
gagacaaattacatcgactacctg

SEQ ID NO: 108 - DNA, 24 bp, Artificial sequence
  Synthetic polynucleotide
  misc_feature: aligned sequences - X89554_Echo27_3D and X89548_Echo19_3D of FIG. 12
gagacaaattacattgactacttg

SEQ ID NO: 109 - DNA, 24 bp, Artificial sequence
  Synthetic polynucleotide
  misc_feature: aligned sequence - X89541_Echo12_3D of FIG. 12
gagacaaactacatcgactaccta

SEQ ID NO: 110 - DNA, 23 bp, Artificial sequence
  Synthetic polynucleotide
  misc_feature: consensus sequence - untitled consensus; aligned sequences - AF465516; Echo_guide2 and X89538_Echo7_3D of FIG. 12
tcccaccacctgtacagagacaa

SEQ ID NO: 111 - DNA, 23 bp, Artificial sequence
  Synthetic polynucleotide
  misc_feature: aligned sequence - X89554_Echo27_3D of FIG. 12
tctcaccatctgtacagagataa

SEQ ID NO: 112 - DNA, 23 bp, Artificial sequence
  Synthetic polynucleotide
  misc_feature: aligned sequence - X89548_Echo19_3D of FIG. 12
tcccaccacctgtacagagataa

SEQ ID NO: 113 - DNA, 23 bp, Artificial sequence
  Synthetic polynucleotide
  misc_feature: aligned sequence - X89541_Echo12_3D  of FIG. 12
tctcaccacctgtacagagacaa

SEQ ID NO: 114 - DNA, 23 bp, Artificial sequence
  Synthetic polynucleotide
  misc_feature: consensus sequence - untitled consensus; aligned sequences - AF465516; Echo_guide3 and X89538_Echo7_3D of FIG. 12
ttcaggatgattgcatatggtga

SEQ ID NO: 115 - DNA, 23 bp, Artificial sequence
  Synthetic polynucleotide
  misc_feature: aligned sequence - X89554_Echo27_3D of FIG. 12
ttcaggatgatagcatatggtga

SEQ ID NO: 116 - DNA, 23 bp, Artificial sequence
  Synthetic polynucleotide
  misc_feature: aligned sequence - X89548_Echo19_3D of FIG. 12
tttaggatgatcgcatatggtga

SEQ ID NO: 117 - DNA, 23 bp, Artificial sequence
  Synthetic polynucleotide
  misc_feature: aligned sequence - X89541_Echo12_3D  of FIG. 12
tttagaatgattgcatatggtga

SEQ ID NO: 118 - DNA, 23 bp, Artificial sequence
  Synthetic polynucleotide
  misc_feature: consensus sequence - untitled consensus of FIG. 12
ttgattatgacaccagcagataa

SEQ ID NO: 119 - DNA, 23 bp, Artificial sequence
  Synthetic polynucleotide
  misc_feature: aligned sequence - AF465516; Echo_guide4 and X89538_Echo7_3D
ttggttatgacaccagcagataa

SEQ ID NO: 120 - DNA, 23 bp, Artificial sequence
  Synthetic polynucleotide
  misc_feature: aligned sequence - X89554_Echo27_3D of FIG. 12
ttgattatgacaccagcagacaa

SEQ ID NO: 121 - DNA, 23 bp, Artificial sequence
  Synthetic polynucleotide
  misc_feature: aligned sequence - X89548_Echo19_3D of FIG. 12
ttgatcatgacaccagcagataa

SEQ ID NO: 122 - DNA, 23 bp, Artificial sequence
  Synthetic polynucleotide
  misc_feature: aligned sequence - X89541_Echo12_3D  of FIG. 12
ctgattatgacaccagcagacaa

(19) United States
(12) Patent Application Publication
(10) Pub. No.: US 2025/0186616 A1
(43) Pub. Date: Jun. 12, 2025

(54) Molecular Systems and Therapies Using the Same

(71) Applicants: AGENCY FOR SCIENCE, TECHNOLOGY AND RESEARCH, Singapore (SG); NATIONAL UNIVERSITY OF SINGAPORE, Singapore (SG)

(72) Inventors: Choong Tat Keng, Singapore (SG); Jang Hann Chu, Singapore (SG); Wei Leong Chew, Singapore (SG); Ching Hua Lee, Singapore (SG)

(21) Appl. No.: 17/912,553

(22) PCT Filed: Mar. 22, 2021
(86) PCT No.: PCT/SG2021/050155

§ 371 Date: Sep. 19, 2022

(30) Foreign Application Priority Data
Mar. 20, 2020 (SG) 10202002623W

(51) Int. Cl. A61K 48/00; C12N 9/22; C12N 15/11; C12N 15/86

(52) U.S. Cl. CPC A61K 48/0058; C12N 9/22; C12N 15/11; C12N 15/86; C12N 2310/20; C12N 2750/14143

(57) ABSTRACT

Disclosed herein are molecular systems comprising an RNA-guided RNA-targeting effector protein, and one or more guide RNA molecule (gRNA). Also disclosed herein are guide RNA molecules for use in the systems disclosed herein, as well as the use of the described systems in the treatment or prevention of disease.

### CROSS-REFERENCE TO RELATED APPLICATIONS

This application claims the benefit of priority of Singapore provisional application No. 10202002623W, filed 20 Mar. 2020, the contents of it being hereby incorporated by reference in its entirety for all purposes.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55794_Substitute_Seq.txt”, which was created on Jun. 28, 2023, and is 55,304 bytes in size. The subject matter of the Sequence Listing is incorporated herein in its entirety by reference.

### FIELD OF THE INVENTION

The present invention relates generally to the field of molecular biology. In particular, the present invention relates to the gene editing technology for the treatment of diseases.

### BACKGROUND OF THE INVENTION

RNA viruses comprise of a significant group of pathogens that pose a constant health threat and which can lead to global outbreaks and pandemics. An example is seen in the recent SARS-COV-2 global pandemic which had brought recession in many of the world economies and restricted global human movement to a minimum, hence it is essential that we continue to explore new therapeutics options to treat RNA viruses to avoid the same situation in the future. The recent SARS-COV-2 pandemic has also shown that conventional therapeutics such as antibodies treatment are ineffective in a global pandemic due to bottlenecks in manufacturing and distribution of such therapeutics and drug therapeutics have shown limited efficacy and usually with undesired side effects.

Enterovirus 71 (EV71), Coxsackievirus (CAV16 and CAV6) and Parechovirus are RNA viruses that are highly contagious and spread through bodily fluids. EV71 infection is most common in children younger than 5 years of age, with about 50-80% of children tested seropositive for EV71, and has also observed in adults, albeit to a lesser extent. Because of the symptoms associated with the infection, EV71 is considered to be a major contributor to the disease known as hand, foot and mouth disease (HFMD). The infection may occasionally result in severe neurological diseases or death. To date, there is no commercially available vaccine or therapeutics for the prevention or elimination of, for example, EV71 infection. Clinical trials are ongoing only for vaccine modalities and not therapeutic modalities. In recent years, EV71 infections have reached record incidence rates in several countries such as but not limited to Singapore, Malaysia and China, and this highlights a pressing need to develop vaccines and therapeutics against the currently incurable infections.

Despite many years of research into the potential of using human intravenous immunoglobulin (IV Ig) and monoclonal antibodies (mAb) as therapeutics, progress has been hampered by concerns and risks of, among others, antibody-dependent enhancements (ADE) or antibody-dependent cell-mediated cytotoxicity (ADCC), as well as technical barriers to humanizing monoclonal antibodies and risk of other pathogen spreading through the use of human intravenous immunoglobulin.

There are currently no commercial vaccines or therapeutics available capable of preventing or treating the diseases disclosed herein. Maintaining good personal hygiene and quarantined of infected individual have been the only measures taken by the public health sector. Thus, there is an unmet need for systems, compositions and methods of detecting and treating the viral diseases disclosed herein.

### SUMMARY OF THE INVENTION

In one aspect, the present disclosure refers to a molecular system comprising (a) an RNA-guided RNA-targeting effector protein, and (b) one or more guide RNA molecule (gRNA); wherein each of the one or more guide RNAs comprise a guide sequence that is at least 70%, at least 80%, at least 90%, at least 95%, or 100% identical to one of the sequences set forth in SEQ ID NO: 1 to 6.

In another aspect, the present disclosure refers to a molecular system comprising (a) an RNA-guided RNA-targeting effector protein, and (b) a collection of at least 4 different guide RNA molecules (gRNAs); wherein at least 4 of the gRNAs in said collection comprise a guide sequence that is at least 70%, at least 80%, at least 90%, at least 95%, or 100% identical to one of the sequences set forth in SEQ ID NO: 1 to 6.

In yet another aspect, the present disclosure refers to a method of treating, preventing, suppressing, and/or alleviating a disease associated with or caused by infection, propagation and/or replication of an RNA virus in a subject, comprising administering to a subject in need thereof a molecular system comprising (a) an RNA-guided RNA-targeting effector protein and/or a polynucleic acid encoding said effector protein, and (b) one or more gRNAs and/or one or more polynucleic acids encoding said one or more gRNAs; wherein each of the one or more gRNAs comprises a guide sequence that is at least 70%, at least 80%, at least 90%, at least 95% or 100% identical to one of the sequences set forth in SEQ ID NO: 1-6.

In a further aspect, the present disclosure refers to a method of treating, preventing, suppressing, and/or alleviating a disease associated with or caused by infection, propagation and/or replication of an RNA virus in a subject, comprising administering to a subject in need thereof a molecular system comprising (a) an RNA-guided RNA-targeting effector protein and/or a polynucleic acid encoding said effector protein, and (b) a collection of at least 4 different gRNAs and/or one or more polynucleic acids encoding said collection of gRNAs; wherein at least 4 of the gRNAs in said collection comprise a guide sequence that is at least 70%, at least 80%, at least 90%, at least 95% or 100% identical to one of the sequences set forth in SEQ ID NO: 1-6.

In one aspect, the present disclosure refers to polynucleotides encoding the molecular system as disclosed herein.

In another aspect, the present disclosure refers to a vector encoding one or more gRNAs, wherein the one or more gRNAs encoded by said vector comprise a guide sequence that is at least 70%, at least 80%, at least 90%, at least 95%, or 100% identical to one of the sequences set forth in SEQ ID NO: 1-6.

In yet another aspect, the present disclosure refers to a vector encoding the molecular system as disclosed herein.

In one aspect, the present disclosure refers to a composition comprising the polynucleotide as disclosed herein, and/or the vector as disclosed herein.

In a further aspect, the present disclosure refers to a method of treating, preventing, suppressing, and/or alleviating a disease associated with or caused by pathogenesis, infection, propagation and/or replication of a virus of the Enterovirus genus in a subject, comprising administering to a subject in need thereof the polynucleotides as disclosed herein, and/or the vector as disclosed herein, and/or the composition as disclosed herein.

In one aspect, the present disclosure refers to a guide RNA molecule (gRNA), comprising a guide sequence that is at least 70%, at least 80%, at least 90%, at least 95%, or 100% identical to any one of the sequences set forth in SEQ ID NO: 1-6.

In another aspect, the present disclosure refers to a guide RNA molecule comprising a guide sequence as set forth in any one of SEQ NO: 1-6.

### BRIEF DESCRIPTION OF THE DRAWINGS

The invention will be better understood with reference to the detailed description when considered in conjunction with the non-limiting examples and the accompanying drawings, in which

FIG. 1 shows a schematic showing the design and construction of plasmid for AAV packaging of CRISPR-CasRx. Plasmid of pZac2.1-CMV-CasRx-3×HA-PolyA-U6-gRNA. Between the ITR sequences, there is a CMV promoter which drives the expression of CasRx sequence and there is a 3×HA tag at the end of the CasRx sequence followed by a rabbit polyadenylation sequence. This is followed by the U6 promoter which drives the transcription of the inserted guide sequences followed by another ITR sequence. Guide sequences can be cloned into this backbone using the BbsI restriction enzyme digestion. Sequence of full plasmid and primers for cloning plasmids are shown in Table 1.

FIG. 2 shows the results of validation experiments showing the expression of CasRx in AAV-CasRx transduced human cells. To validate that the packaged viruses are able to transduce mammalian cells and express the CasRx cargo, 1×10⁵ human immortalized corneal endothelial cells, B4G12, were transduced and plated in a 48-well plate at MOI 100K. Anti-HA staining is carried out at 1:200 for 2 hours and anti-GFP at 1:1500 for 2 hours. Secondary antibodies staining is carried out at 1:1000 for 2 hours. Images are taken at exposure 15 milliseconds and gain set at 6400 for bright field and the 488 channel, exposure is set at 800 us and gain at 6400 for the DAPI channel. The results showed that AAVDJ-eGFP is able to transduce B4G12 cells and specific signal of eGFP is detected compared to no transduction control (FIG. 2, row 1 and 2 respectively). The results also confirmed the expression of the CasRx-3×HA protein in the AAVDJ-CMV-CasRx transduced human B4G12 cells (FIG. 2, third row) and that the signal for both the primary and secondary antibodies are specific as shown by the low background signal in the non-infected control (FIG. 2, fourth row).

FIG. 3 shows an alignment of the position of the guides designed against the GFP gene sequence. Two RNA guides were designed for target GFP gene sequence closer to the 5′ end, with the logic that cleavage at the earlier bases would terminate translation more efficiently since protein translation starts from the 5′ end (FIG. 3). GFP knockdown will be performed using these guides to validate the RNA cleavage activity of CasRx. Primers for guides cloning are shown in Table 1. GFP guide1 sequence-gtgaacagctcctcgcccttgct (SEQ ID NO: 7); GFP guide2 sequence-gctgaacttgtggccgtttac (SEQ ID NO: 8).

FIG. 4 shows an alignment of the position of the guides designed against the EV71 3D protein gene sequence. Six RNA guides were designed for target EV71 3Dpro gene sequence based on highly conserved regions (>85% similarity) from the alignment of six EV71 strains (H8-1, S41, Sin002209, MZ, NJ2017iso2, Shenzhen001-2006) using Benchling Clustal Omega alignment and Lasergene Seqman Pro 15 (DNAStar). Guides sequence and positions with respect to strain41 3Dpro gene sequence are shown in the figure above. Primers for cloning guides are shown in Table 1. Alignments are shown in FIGS. 10 to 12. EV71_3D guide1 sequence-gttggtccattgatgtttagtct (SEQ ID NO: 1); EV71_3D guide2 sequence-ttggaaaacagggcttgttcaaa (SEQ ID NO: 2); EV71_3D guide3 sequence-tagcacgcttcctccatgctcat (SEQ ID NO: 3); EV71_3D guide4 sequence-tatttatccatgtagaatttcat (SEQ ID NO: 4); EV71_3D guide5 sequence-atgatgttgttgatcattgaatt (SEQ ID NO: 5); EV71_3D guide6 sequence-tctgcaggagtcatggtcaaaccatactc (SEQ ID NO: 6).

FIG. 5 shows the results of a biopanning of AAV serotypes to identify AAV that efficiently transduce human rhabdomyosarcoma (RD) cells. The human skeletal muscle was proposed to be a target organ that supports EV71 persistent infection and replication. Using the human rhabdomyosarcoma (RD) cell line (ATCC), an AAV panel biopanning experiment was performed to determine the most suitable AAV serotype(s) that can efficiently transduce human muscle cells to deliver the CRISPR-Cas tools for viral RNA targeting. 10K cells were plated into each well of a 96-well plate and transduced with each serotype of AAV at MOI of 100K and the expression of GFP protein was quantitated at 72 hours post-transduction. Experiments were performed in duplicates. The result showed that AAV1, AAV2 and AAVDJ can transduce human rhabdomyosarcoma (RD) cells more efficiently than other AAV serotypes tested, in the order of increasing efficiency as follow, AAV1, AAVDJ, and AAV2 respectively.

FIG. 6 shows results of the validation of knockdown of GFP expression in AAV-CasRx-GFPguides transduced human rhabdomyosarcoma (RD) cells. Human muscle immortalized cell line, RD, were transduced with AAV2-GFP at MOI 10K for expression of GFP. The GFP knockdown efficiency of guide1 only, guide2 only and guide1+guide2 are tested by transduction of the GFP-expressing rhabdomyosarcoma (RD) cells with AAV2-CasRx bearing the guides at MOI 100K (left figure) and MOI 1000K (right figure). The result showed that the knockdown of GFP by individual guide is present but efficiency of knockdown is increased by pooling the two guides. Comparison between two groups was analysed by Student t test (two-tailed) using the software Prism 8. * p<0.05.

FIG. 7 shows the results of the inhibition of EV71 replication in AAV-CasRx-EV71_3Dguides transduced EV71-infected human rhabdomyosarcoma (RD) cells. Proof-of-concept experiment for the ability of AAV-CRISPR-CasRx-EV71_3Dguides to inhibit EV71 viral replication. In a 96-well plate, 10×10³ rhabdomyosarcoma (RD) cells were seeded in each well and transduced with AAVDJ-eGFP with no guides or AAVDJ-CasRx bearing either GFP guide2 or EV71 guide(s) for 72 hours and then subjected to EV71 infection at MOI of 1 for 12 h, the supernatant is then harvested for virus plaque assay. The result from the plaque assay counts suggest that individual guide have little inhibitory effect at MOI 100K but pooling the guides (six guides) together can have a strong inhibitory effect on EV71 replication of up to 3 logs of magnitude of virus titre reduction at AAV MOI of 100K. The control using no guides or GFP guide also suggest that the inhibitory effect is specific to the EV71 guides. Comparison between two groups (CasRx-eGFPg2 and CasRx-EV71_3Dguide(s)) was analysed by Student t test (two-tailed) using the software Prism 8. * p<0.05.

FIG. 8 shows the results of inhibition of EV71 replication in EV71-infected human rhabdomyosarcoma (RD) cells transduced with different number of CasRx EV71 3Dpro gene targeted guides. Proof-of-concept experiment for the ability of AAV-CRISPR-CasRx-EV71_3Dguides to inhibit EV71 viral replication. In a 96 well plate, 10×10³ rhabdomyosarcoma (RD) cells were seeded in each well and transduced with AAV2-eGFP with no guides or AAV2-CasRx bearing either GFP guide2 or EV71 guide(s) for 72 hrs and then subjected to EV71 infection at MOI of 1 for 12 h, the supernatant is then harvested for virus plaque assay. The result from the plaque assay counts suggest that all six individual guide have strong inhibitory effect of up to 1 log virus titre reduction at MOI 1000K but pooling the guides gradually together can have an increasing inhibitory effect on EV71 replication of up to 4 logs of magnitude of virus titre reduction at AAV2 MOI of 1000K for the 6 guides pool. The exponential increase in inhibitory effect is stronger in the 4, 5 and 6 guides pool, suggesting either a critical number of guides targets or a synergy between the guides within the pool are important to achieve higher inhibitory efficacy. The control using no guides or GFP guide2 also suggest that the inhibitory effect is specific to the EV71 guides. Comparison between two groups (CasRx-eGFPg2 and CasRx-EV71_3Dguide(s)) was analysed by Student t test (two-tailed) using the software Prism 8. *p<0.05.

FIG. 9 shows a schematic of the technology disclosed herein, based on the example of EV71 RNA. In the first step, CRISPR-CasRX components delivered into target cells (in one embodiment, via adeno-associated viruses), resulting in the expression of CasRX and guide RNA(s) in EV71-infected cells. In a next step, the combination of a guide RNA with CasRX results in cleavage of, for example, the EV71 RNA genome to disrupt replication and viral functions, as well as cleavage of, for example, EV71 mRNA (e.g. 3Dpol gene) to disrupt replication and viral functions. It is noted that a multitude of CRISPR-Cas-gRNA complexes can be used within the same cell and/or on the same viral RNA molecule, despite only one exemplary CRISPR complex shown here for clarity.

FIG. 10 shows the alignment sequences of strain H8-1 to other Enterovirus strains. The sequences shown in FIG. 10 are present in Table 1.

FIG. 11 shows the alignment sequences of LC126150, CAU05876, and an untitled consensus. The sequences shown in FIG. 11 are present in Table 1.

FIG. 12 shows the alignment sequences of an Echovirus (Parechovirus) consensus sequence strain to other Echovirus (Parechovirus) sequences. The sequences shown in FIG. 12 are present in Table 1.

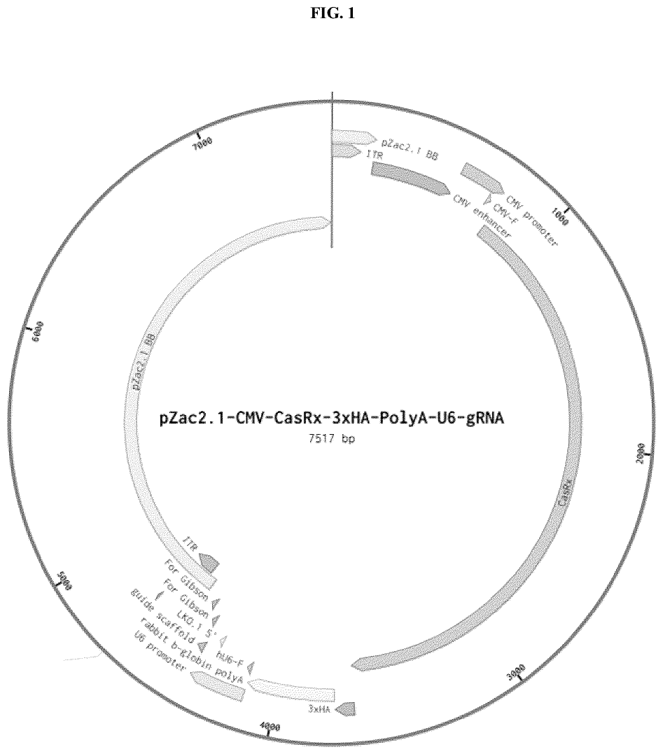

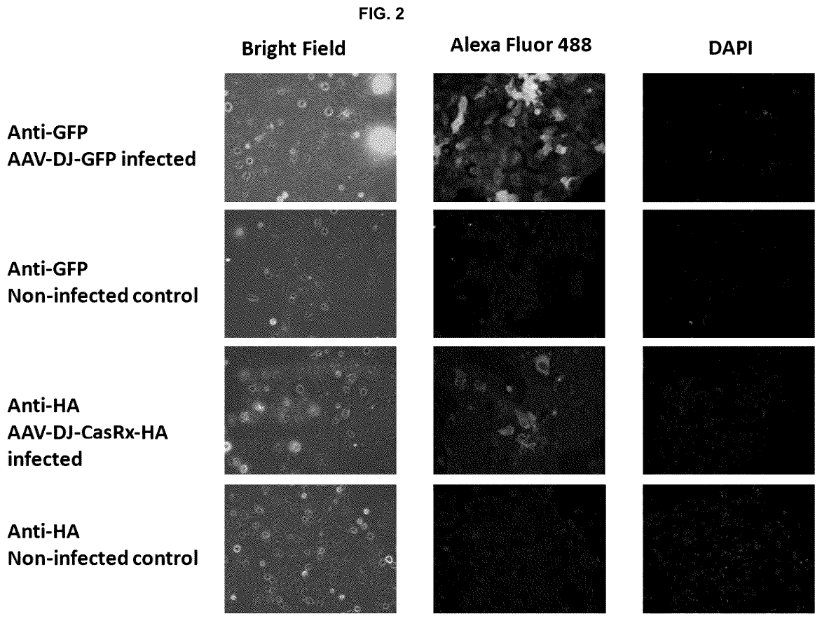

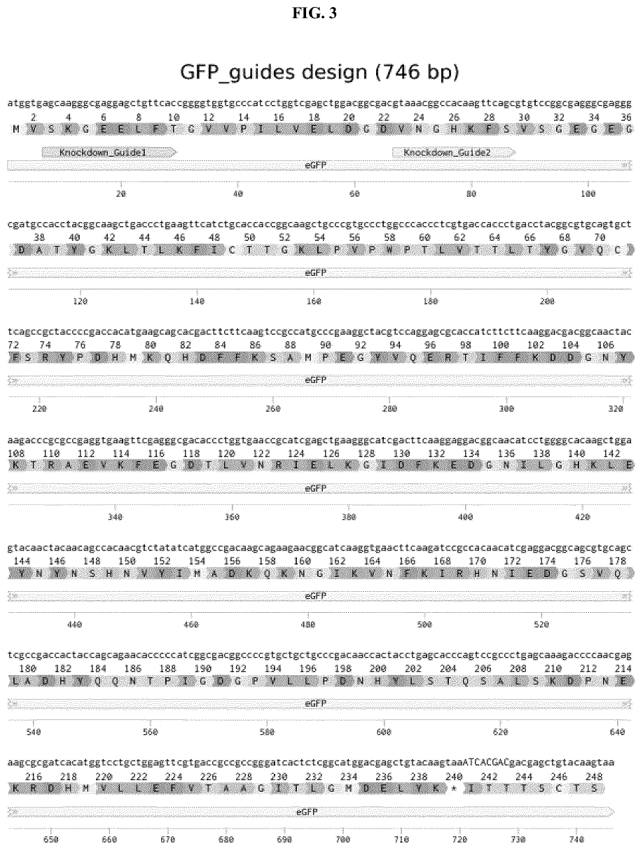

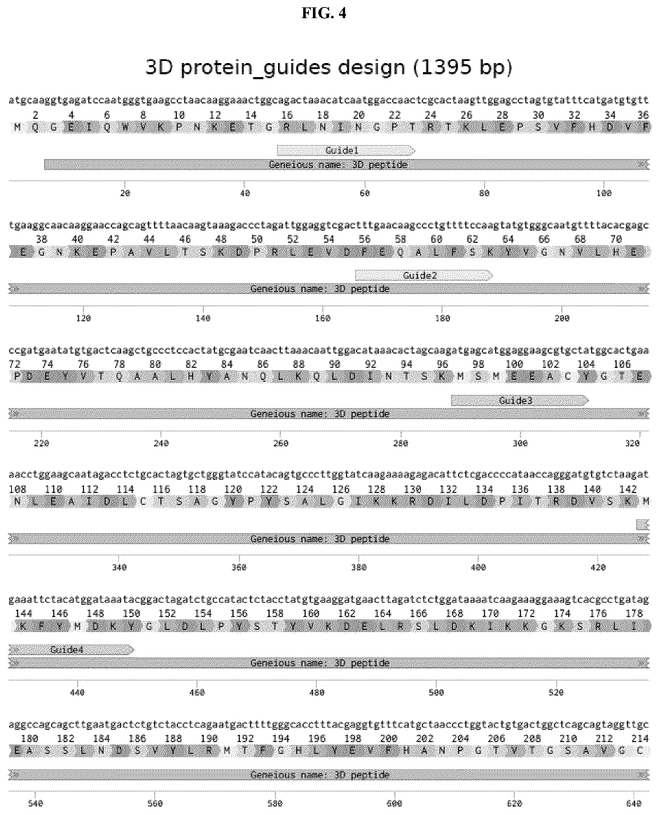

### Definitions

As used herein, the term “biopanning” refers to the identification or selection of the best-performing AAV serotype(s) amongst a larger library of different AAV serotypes.

As used herein, the term “RNA-guided RNA-targeting effector protein” refers to a protein which is capable of forming a RNA-targeting complex with a guide RNA or gRNA, said complex is capable of binding with and effecting changes on one or more RNA target molecules. The changes effected on the RNA targets refer to any chemical or physical changes to the components or structure of the RNA molecule, which may include but are not limited to: breaking/cleaving the polynucleotide, substituting one or more nucleotide bases, and inserting or deleting one or more nucleotide bases. In one example, the RNA-guided RNA-targeting effector protein is a Cas protein. In another example, the Cas protein is a Cas nuclease.

The term “guide RNA” or “gRNA” refers to an RNA molecule comprising a sequence (guide sequence) sufficient complementarity with a target RNA sequence to hybridize with the target RNA sequence and direct sequence-specific binding of an RNA-targeting complex to the target RNA sequence. In some embodiments, the degree of complementarity between a guide sequence (within a guide RNA) and its corresponding target RNA sequence, when optimally aligned using a suitable alignment algorithm, is about or more than about 60%, 70%, 80%, 85%, 90%, 95%, 97.5%, 99%, or 100%.

As used herein, the term “CRISPR-Cas” refers to genome editing technology based on the capability of clustered regularly interspaced palindromic repeats (CRISPR) and, for example, the CRISPR-associated protein nuclease to induce, for example, double-strand (ds) DNA breaks in a specific location that is complementary to the synthetic guide RNA (sgRNA) sequence integrated into the CRISPR-Cas complex/system. This allows the deletion, addition, and/or modification of genes and/or other genomic elements, such as, but not limited to, transcription elements, promoters, promoter enhancers, transcription enhancers, restriction sites, mutations, selection markers, for example antibiotic selection cassettes, and the like. Such nuclease can be isolated from, for example, Streptococcus pyogenes (in the case of Cas9).

The functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. Three types of CRISPR mechanisms have been identified so far, of which type II is the most well studied. Also, other combinations of CRISPR, for example CRISPR-Cpf1 have been developed. Also contemplated herein is the use of CRISPR technologies, wherein the Cas proteins or functionally analogue proteins are not isolated from S. pyogenes. Examples of Cas proteins are, but are not limited to, Cas nucleases (for example Cas9 and Cas13 proteins), or proteins with the same functionality, isolated from S. pyogenees, Staphylococcus aureus, or any representatives of the archaea kingdom. Cas9 proteins can also be substituted with so-called CasX and CasY proteins. In another example, examples of Cpf1 proteins, or proteins with the same functionality are isolated from, but are not limited to, Acidaminococcus sp. and Lachnospiraceae. In terms of adaptive immunity, the mechanism of CRISPR-Cas-mediated defence is as follows: invading DNA from viruses or plasmids is cut into small fragments and incorporated into a CRISPR locus amidst a series of short repeats (around 20 base pairs). The loci are transcribed, and transcripts are then processed to generate small RNAs (crRNA-CRISPR RNA; also referred to as synthetic guide RNA (sgRNA) in an in vitro setting), which are used to guide effector endonucleases that target invading DNA based on sequence complementarity. In terms of gene editing, the CRISPR-Cas system works according to the same principle, with the sgRNA guiding the effector nucleases to the desired sections of the DNA, in which the excision is to be made.

The term “vector” as used herein refers to a macromolecule or association of macromolecules that comprises or associates with a polynucleotide and which can be used to mediate delivery of the polynucleotide to a cell. Illustrative vectors include, for example, plasmids, viral vectors (virus or the viral genome thereof), liposomes, and other gene delivery vehicles.

As used herein, the term “AAV” refers to an Adeno-associated virus. The term AAV may be used to refer to the virus itself, or derivatives thereof, for example, but not limited to, the viral capsid, the viral genome, viral particles, viral fragments and combinations thereof. The term “AAV” encompasses all subtypes, both naturally occurring and recombinant forms, and variants thereof except where required otherwise. Naturally occurring forms of AAV refer to any adeno-associated virus or derivative thereof comprising a viral capsid that consists of viral capsid proteins that occur in nature. Non-limiting examples of naturally occurring AAV include AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV9, AAV10, AAV11, AAV12, AAV13, rh10, avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc. “Recombinant AAV”, or “rAAV” includes any AAV that comprises a heterologous polynucleotide sequence in its viral genome. Other examples of AAV serotypes and variants useful for use as vectors include, but are not limited to, AAVDJ, AAV-PHP.S, AAV-PHP.B, AAV-PHP.eB, and Anc80.

As used herein, the term “Enterovirus 71” (EV71), also known as Enterovirus A71 (EV-A71), refers to a virus of the genus Enterovirus in the Picornaviridae family, notable for its role in causing epidemics of severe neurological disease and hand, foot, and mouth disease (HFMD) in children. Enterovirus 71 has also been reported to infrequently cause polio-like syndrome permanent paralysis.

As used herein, the term “Coxsackievirus” refers to a small number of related enteroviruses that belong to the Picornaviridae family of non-enveloped, linear, positive-sense single-stranded RNA viruses, as well as its genus Enterovirus, which also includes poliovirus and echovirus. Enteroviruses are among the most common human pathogens. Ordinarily, its members are transmitted by the faecal-oral route. Coxsackieviruses share many characteristics with poliovirus. Coxsackieviruses are also known to be among the leading causes of aseptic meningitis, along with echovirus and mumps virus.

As used herein, the term “Parechovirus”, also referenced as “Echovirus”, refers to a polyphyletic group of viruses associated with enteric disease in humans. The name is derived from “enteric cytopathic human orphan virus”. These viruses were originally not associated with disease but many have since been identified as disease-causing agents. The term “echovirus” was used in the scientific names of numerous species. Thus, the term “Echovirus” has been replaced with the term “Parechovirus” to denote viruses belonging to this sub-species of Picornaviridae family, while other viruses previously considered to be Echoviruses have been reassigned to other species, also within the Picornaviridae family. Nevertheless, all echoviruses are now recognized as strains of various species, most, if not all, of which are in the Picornaviridae family. It is noted that the terms “Echovirus” and “Parechovirus” have been used interchangeably herein. Examples, of a Parechovirus are, but are not limited to, Parechovirus A, Parechovirus B, Parechovirus C, Parechovirus D, Parechovirus E, and Parechovirus F.

As used herein, the term “hand, foot, and mouth disease” (HFMD) refers to a febrile disorder usually caused by Coxsackievirus A16, Enterovirus 71, or other enteroviruses. Infection causes a vesicular eruption on the hands, feet, and oral mucosa. Atypical HFMD due to Coxsackievirus A6 often causes high fever with papulovesicular lesions progressing to vesicobullous lesions and bullae that are widely distributed on the body. Viruses causing HFMD are of the Picornaviridae family, with Coxsackievirus A16 is the most common cause of HFMD. Enterovirus 71 (EV-71) is the second-most common cause. Many other strains of Coxsackievirus and enterovirus can also be responsible.

As used herein, the term “transduction or “transduced” refers to a vector-mediated gene transfer procedure, which is usually used to describe both bacteria and/or virus-mediated methods.

### DETAILED DESCRIPTION OF THE PRESENT INVENTION

Enterovirus 71, Coxsackie CAV16 and CAV6, and Parechoviruses are viruses belonging to the Picornaviridae family, which by definition consists of non-enveloped, positive, single-stranded RNA viruses. These viruses are most well-known for their manifestation in young children as hand, foot and mouth disease (HFMD), presenting with blisters or lesions observable in the oral cavity, and with lesions or blisters occasionally occurring on the palms of the hand and the soles of the feet. In more severe cases, hand, foot and mouth disease also presents with more severe symptoms, such as but not limited to, paralysis, aseptic meningitis and encephalitis or even death.

There are currently no commercial vaccines or therapeutics available which are capable of preventing or treating the diseases disclosed herein. Maintaining good personal hygiene and quarantined of infected individual have been the only measures taken by the public health sector.

Thus, disclosed herein is a method of treating, preventing, suppressing, and/or alleviating a disease associated with or caused by pathogenesis, infection, propagation and/or replication of a virus of the Enterovirus genus in a subject. In another example, the method disclosed herein comprises administration to a subject in need thereof the polynucleotides of as disclosed herein, and/or the vector as disclosed herein, and/or the composition as disclosed herein.

CRISPR-Cas presents itself as an alternative in antiviral therapeutics development. CRISPR stands for clustered regularly interspaced short palindromic repeats, and was first discovered in the bacterial immune system. However, it was not until 2012 that the CRISPR-Cas system was found to have the capability of precisely editing DNA in eukaryotes. Before the emergence of CRISPR, research efforts had been focused on mainly the development of humanized monoclonal antibodies targeting neutralizing epitopes as antivirals since the success story of Herceptin, an anti-HER2 humanized monoclonal antibody used for the treatment of breast cancer. Traditional drug screenings to short-list for compounds or small molecules are also popular approaches for therapeutics development; however, these screening methods often have an inhibitory effect on viral replication. Despite drug screening and monoclonal antibodies studies, there are currently no prospective ongoing clinical trials nor any commercialized therapeutics for HFMD treatment.

The use of CRISPR-Cas has been limited by an efficient delivery system in vivo, and current FDA approved drugs were based on adeno-associated virus (AAV) delivery, which is considered safe for human applications as it does not elicit an acute inflammatory immune response. However, the maximum cargo size of adeno-associated virus is roughly 4.7 kilo base pairs (kbp), which is insufficient for the delivery of most known Cas enzymes, as these usually exceed the stated limit, especially after cloning in the mammalian promoter sequence.

Thus, the molecular systems disclosed herein can be comprised or encoded by a polynucleic acid encoding said effector protein and/or the one or more polynucleic acids encoding said gRNAs are comprised in one or more vectors. As used herein, the term “vector” refers to any molecule (for example, but not limited to, nucleic acid, plasmid, or virus) used to transfer coding information into a host cell.

As used in the art, viral vectors are tools used to deliver genetic material into cells. This process can be performed inside a living organism (in vivo) or in cell culture (in vitro). Specifically, viruses have evolved specialized molecular mechanisms to efficiently transport their genomes inside the cells they infect. Delivery of genes or other genetic materia by a vector is termed transduction and the infected cells are described as transduced. In addition to their use in molecular biology research, viral vectors are used for gene therapy and the development of vaccines.

Thus, in one example, the vector is a viral vector. In another example, the viral vector is, but is not limited to, an adenoviral vector, an Adeno-associated virus (AAV) vector, a lentiviral vector, or a retroviral vector.

By way of an example of a vector used in the presently disclosed application, the adeno-associated virus (AAV) is a small virus that infects humans and some other primate species. The adeno-associated virus is not currently known to cause disease, and upon infection, has been shown to cause only a mild immune response. The adeno-associated virus is capable of infecting both dividing and non-dividing cells and may incorporate its genome into that of the host cell. Moreover, the adeno-associated virus mostly stays as episomal (that is to say, it can replicate within a host without incorporation of its payload into the host chromosome); performing long and stable expression. These features make the adeno-associated virus a suitable candidate for creating viral vectors for gene therapy. Thus in one example, the viral vector is an adeno-associated virus (AAV) vector. In another example, the adeno-associated virus (AAV) vector is but is not limited to, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV9, AAV10, AAV11, AAV12, AAV13, rh10, AAVDJ, AAV-PHP.S, AAV-PHP.B, AAV-PHP.eB, and Anc80. In one example, the adeno-associated virus (AAV) vector is an AAV2, or AAVDJ, or AAV1 vector.

In one example, there is disclosed a vector encoding one or more gRNAs as disclosed herein. In such an example, the one or more gRNAs encoded by said vector comprise a guide sequence that is at least 70%, at least 80%, at least 90%, at least 95%, or 100% identical to one of the sequences set forth in SEQ ID NO: 1-6. In another example, the vector encodes at least 4 different gRNAs, wherein each of the gRNAs comprises a guide sequence that is set forth in SEQ ID NO: 1-6. In a further example, the vector encodes at least 6 different gRNAs, wherein each of the gRNAs comprises a guide sequence that is set forth in SEQ ID NO: 1-6.

Further disclosed herein are polynucleotides encoding the molecular system as disclosed herein, as well as polynucleotides encoding components required by the molecular system or composition disclosed herein.

Using bioinformatics to mine for Cas enzymes, it was previously found the CasRx, the smallest type VI CRISPR-Cas enzyme, has high RNA-guided RNA-targeting activity. In one example, the CRISPR-Cas modality used herein belongs to the Type VI-D Cas family which is a RNA-guided RNA-targeting Cas modality. This enables delivery of the CRISPR-Cas system into mammalian cells in vivo, via an adeno-associated virus (AAV) delivery system, which in turn allows for RNA targeting of the (foreign) viral RNA genome for cleavage.

Thus, in one example, the RNA-guided RNA-targeting effector protein is an RNA-guided RNA-targeting Cas protein, or a modified variant thereof. In another example, the method or molecular system disclosed herein comprises a RNA-guided RNA-targeting effector protein, wherein the RNA-guided RNA-targeting effector protein is an RNA-guided RNA-targeting Cas protein, or a modified variant thereof.

With regard to the safety profile of CRISPR-Cas technologies, these technologies have been proven to be safe for use in, for example, gene therapies and CRISPR-Cas therapeutics. This is also the result of higher specificity of CRISPR-Cas technologies compared to other gene editing methods known in the art. It is of note that any RNA editing resulting from type IV CRISPR-Cas used in the present disclosure is not permanent and will terminate once the exogenous DNA has degraded, or after the cell divides. The highly specific activity of the CRISPR-Cas therapeutics will also avoid common issues of “side-effects” from drug compound or small molecules usage or antibody-dependent enhancement (ADE) or antibody-dependent cellular cytotoxicity (ADCC) issues as seen in antibody therapies.

It is of note that, in the art, a therapeutic drug that can precisely target for the pathogen genomic material for destruction has yet to be found. The application of the CRISPR-Cas in antiviral therapeutics shows its effect by eliminating viral replication by directly breaking down the viruses itself. Due to the direct-targeting mechanism, these therapeutics do not rely on the immune system to assist in eliminating the viruses, and can therefore be used with equal potency in patients that are immune-compromised or in young and aged patients with weaker immunity.

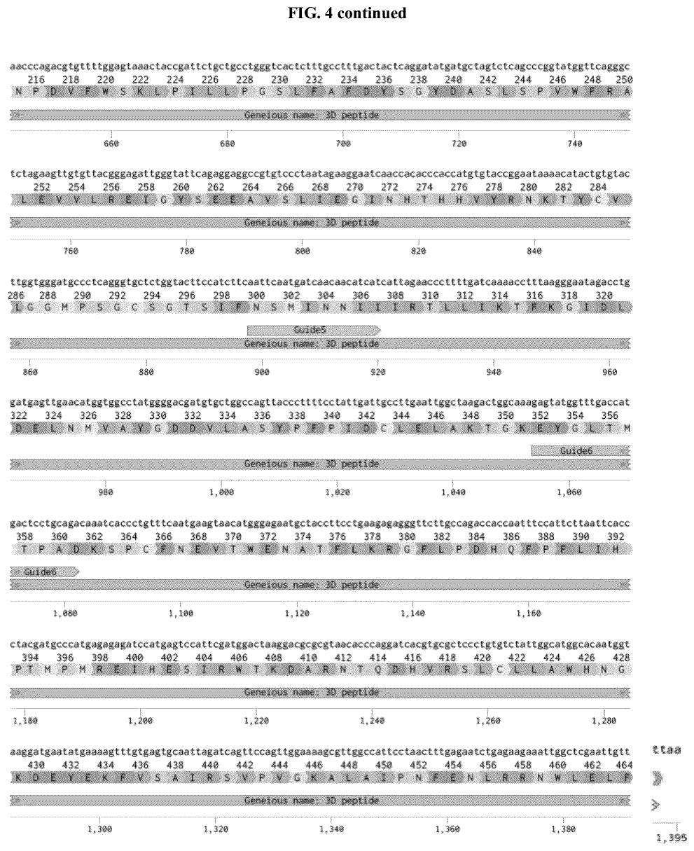

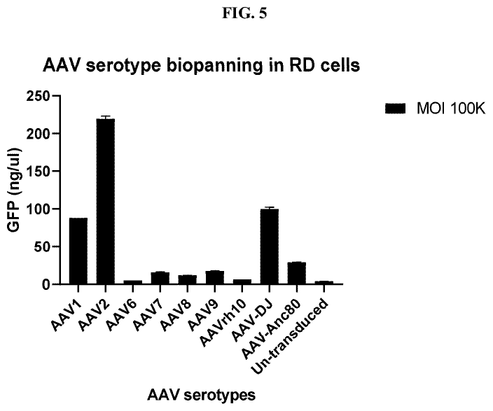

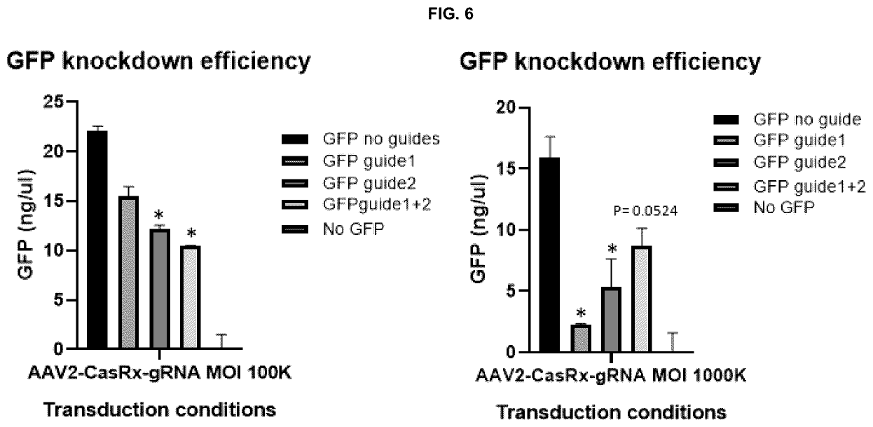

The guide RNAs (gRNAs) as disclosed herein are based on a highly conserved region with a similarity of at least 85% sequence identity between six Enterovirus 71 (EV71) strains (FIG. 4). The guide RNAs disclosed herein are capable of targeting multiple strains. This conservation of sequences further implies that these sequences are of functional importance to the viruses. Without being bound by theory, it is thought that less conserved sequences imply the ability of viruses strains to mutate at those (less conserved sites), thereby escaping CRISPR targeting. Thus, conserved regions of the circulating pathogenic strains are targeted by the (molecular) systems, compositions and uses of the compositions disclosed herein. Specifically, in one example, the target chose was the functionally important polymerase 3D gene.

Thus, disclosed herein is a molecular system based on the CRISPR-Cas system that is capable of targeting RNA. Such a molecular system comprises, for example, (a) an RNA-guided RNA-targeting effector protein, and (b) one or more guide RNA molecule (gRNA). The person skilled in the art would appreciate that the guide RNAs can be customised and tailored to the RNA target that the molecular system is to affect.

In one example, the molecular system disclosed herein is for use in treating a disease associated with or caused by the infection, propagation and/or replication of an RNA virus.

Also disclosed herein are specific guide RNAs (gRNAs) with defined sequences which have been empirically validated to have antiviral activity. In one example, the guide strains are as disclosed herein.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

Thus, also disclosed herein is a guide RNA molecule (gRNA), comprising a guide sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth herein. In one example, the sequence identity of the guide RNA molecules is in comparison to the SEQ ID Nos 1-6 as disclosed herein. Also disclosed herein is a guide RNA molecule comprising a guide sequence as set forth in any one of SEQ NO: 1-6.

In another example, the one or more guide RNAs comprise a guide sequence that is at least 70%, at least 80%, at least 90%, at least 95%, or 100% identical to one of the sequences set forth in SEQ ID Nos 1 to 6. In another example, the guide RNAs are as set forth in SEQ ID Nos to 1 to 6.

It is further disclosed in the scope of the present disclosure that multiple RNAs can be targeted by using a combination or collection of multiple guide RNA molecules, wherein the number of guide RNA molecules is the same as the number of RNA targets that the user wishes to target.

In in one example, the molecular system disclosed herein comprises a collection of at least 4 different guide RNA molecules (gRNAs). This collection of guide RNAs are capable of targeting the same or different targets on the viral RNA, whereby the target is defined by the sequence of the guide RNA. In one example, at least 4 of the gRNAs in said collection comprise a guide sequence that is at least 70%, at least 80%, at least 90%, at least 95%, or 100% identical to one of the sequences set forth in SEQ ID NO: 1 to 6. In another example, the collection comprises at least 4 of the gRNAs disclosed herein, wherein the gRNAs comprise a guide sequence that is at least 70%, at least 80%, at least 90%, at least 95%, or 100% identical to one of the sequences set forth in SEQ ID NO: 1 to 4. In another example, the collection disclosed herein comprises at least 6 different gRNAs, wherein 6 of the gRNAs in said collection comprise a guide sequence that is at least 70%, at least 80%, at least 90%, at least 95%, or 100% identical to one of the sequences set forth in SEQ ID NO: 1 to 6.

This disclosure describes a technology and strategy for targeting and eliminating foreign RNA, for example RNA viruses, using type VI CRISPR-Cas systems programmed to directly cleave the foreign RNA (for example, RNA viral genomes).

Also disclosed herein are (molecular) systems, compositions and uses of the compositions described herein in neutralising a virus infection. Furthermore, the (molecular) systems, compositions and uses of the compositions described herein and also be used to reduce viral replication and thus reduce viral titre in a subject.

It is understood that a subject being infected with a virus may not necessarily display symptoms of the disease that the virus is known to cause. Such subjects are known as viral carriers, and are usually asymptomatic. In such subject, the (molecular) systems, compositions and uses of the compositions described herein are used to reduce viral titres or suppress viral replication, thereby preventing manifestation of disease symptoms in the subject.

Also disclosed herein are (molecular) systems, compositions and uses of the compositions described herein in for the treatment and/or prevention of hand, foot, and mouth disease (HFMD). Also disclose herein is the use of the disclosed composition and molecular systems in the reduction of the amount of viral RNA in the subject. This viral RNA can be, but is not limited to, viruses that cause hand, foot, and mouth disease (HFMD). In one example, the viruses causing hand, foot, and mouth disease (HFMD) is, but are not limited to, Enterovirus 71 (for example, EV71), Coxsackievirus (for example, CAV16 and CAV6), Parechovirus (formerly referred to as Echovirus, including, but not limited to Parechovirus A, Parechovirus B, Parechovirus C, Parechovirus D, Parechovirus E, and Parechovirus F), and combinations thereof.

The viral genome is deactivated by cleavage of the viral RNA genome using the CRISPR-Cas system as disclosed herein. In another example, the Cas protein or nuclease is Cas13 or Cas13d. In one example, the Cas13d nuclease is isolated from Ruminococcus flavefaciens (CasRX). In another example, Cas13d is the Cas13d orthologue of Ruminococcus flavefaciens. The type VI CRISPR-Cas systems target only the RNA viral genomes without risk of undesired activity against the host DNA genome.

Thus, based on the disclosure herein, in one example, the wherein the RNA-guided RNA-targeting effector protein is an RNA-guided RNA-targeting Cas protein, or a modified variant thereof. In another example, the Cas protein is but is not limited to, a Cas13a, a Cas13b, a Cas13c, and a Cas13d protein.

It is noted that in the context of the present disclosed, the claimed system would not function as intended with Cas9 as a nuclease, as Cas9 is a DNA-targeting modality. The application disclosed herein requires a RNA-targeting nuclease for direct targeting and cleavage of the viral genome. Conversely, DNA viruses (such as, for example, herpesviruses, poxviruses) can be targeted by Cas9, but not by Cas13. Thus, in one example, the Cas nuclease is one which targets RNA.

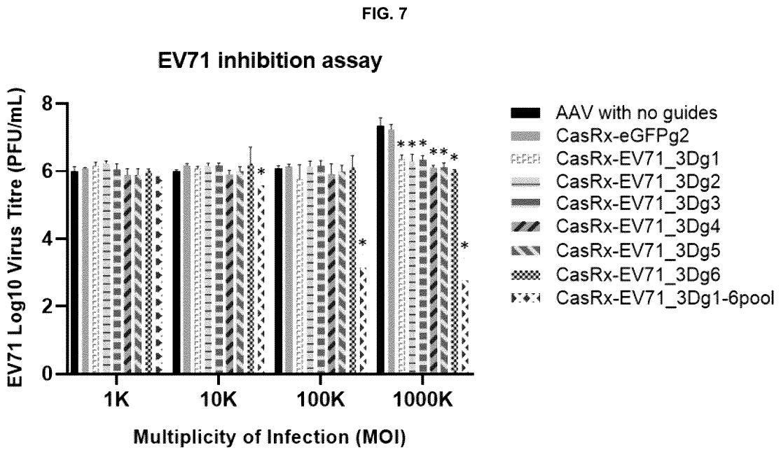

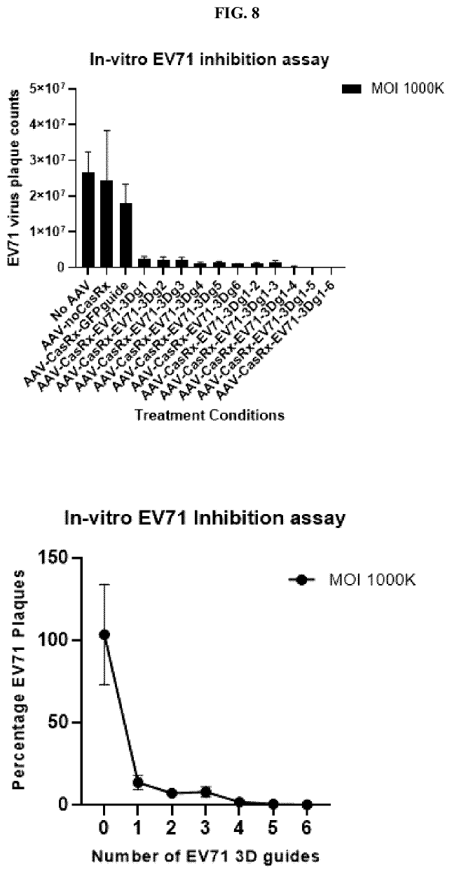

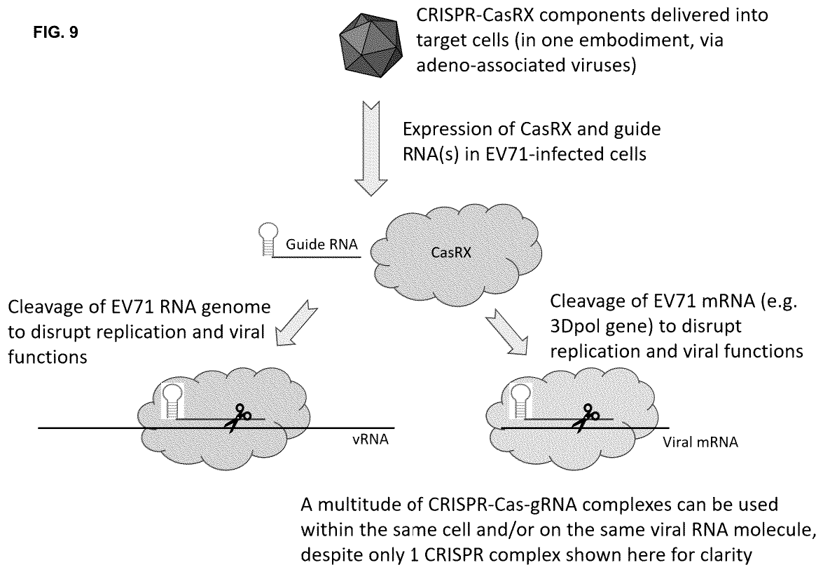

Also disclosed herein is a set of guide RNAs (gRNAs) against Enterovirus 71 (EV71), Coxsackievirus and Parechovirus (Echovirus) viral genomes. Use of these guide RNAs in the claimed system has been shown to eliminate the target viruses. This has been shown by the more than 1000× reduction of viral titres in infected cells, when compared to individual gRNAs that do not show antiviral activity when used individually. Exemplary data for the reduction of viral titres in infected cells is shown in FIG. 7, showing data based on the example of Enterovirus 71 (EV71).

In one example, the molecular system as disclosed herein comprises a Cas protein, wherein the Cas protein is a CasRx; and a collection comprises 6 different guide RNA molecules (gRNAs), wherein each of the gRNAs comprised in the collection is a guide sequence that is set forth in SEQ ID NO: 1 to 6.

The disclosure also describes compositions that function both as a prophylactic, that is to say, as compound that prevents primary infection, and as a therapeutic (meaning that the composition eliminates or reduces an existing infection.

In one example, there is disclosed a molecular system as disclosed herein for use in therapy. In another example, there is disclosed a method of treating, preventing, suppressing, and/or alleviating a disease associated with or caused by infection, propagation and/or replication of an RNA virus in a subject. Further disclosed herein is a composition as disclosed herein for use in therapy.

As disclosed herein, the methods of treatments disclosed herein can comprise administering to a subject in need thereof a molecular system comprising (a) an RNA-guided RNA-targeting effector protein and/or a polynucleic acid encoding said effector protein, and (b) one or more gRNAs and/or one or more polynucleic acids encoding said one or more gRNAs; wherein each of the one or more gRNAs comprises a guide sequence that is at least 70%, at least 80%, at least 90%, at least 95% or 100% identical to one of the sequences set forth in SEQ ID NO: 1-6. In another example, the methods of treatment disclosed herein can comprise administering to a subject in need thereof a molecular system comprising (a) an RNA-guided RNA-targeting effector protein and/or a polynucleic acid encoding said effector protein, and (b) a collection of at least 4 different gRNAs and/or one or more polynucleic acids encoding said collection of gRNAs; wherein at least 4 of the gRNAs in said collection comprise a guide sequence that is at least 70%, at least 80%, at least 90%, at least 95% or 100% identical to one of the sequences set forth in SEQ ID NO: 1-6.

The present disclosure also includes use of any one of the following in the manufacture of a medicament for treatment of a disease disclosed herein, the following being the molecular system, the vectors, the compositions, or the guide RNA molecules as disclose herein, or combinations thereof.

In the context of this specification, the term “treatment”, refers to any and all uses which remedy a disease state or symptoms, prevent the establishment of disease, or otherwise prevent, hinder, retard, or reverse the progression of disease or other undesirable symptoms in any way whatsoever.

Thus, also disclosed herein is a composition comprising the polynucleotide as disclosed herein and/or the vector as disclosed herein. Such a composition as disclosed herein includes within its scope a pharmaceutically acceptable composition. The pharmaceutical composition is therefore suitable for use in the methods of treatment disclosed herein, as well as being suitable for use in the manufacture of a medicament as disclosed herein.

In one example, such a composition as disclosed herein further comprises an RNA-guided RNA-targeting effector protein. Such a guide effector protein can be chosen from the RNA-targeting effector proteins disclosed herein, such as for example, but not limited to Cas13a, Cas13b, Cas13c, and Cas13d.

In the context of this specification the terms “therapeutically effective amount” and “diagnostically effective amount”, include within their meaning a sufficient but non-toxic amount of a compound or composition of the invention to provide the desired therapeutic or diagnostic effect. The exact amount required will vary from subject to subject depending on factors such as the species being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

It is shown in the data disclosed herein that viral replication (of, for example, the viruses disclosed herein) is inhibited via direct targeting of the viral RNA genome for cleavage.

Disclosed herein is a DNA construct that comprising: a promoter that drives transgene expression in mammalian cells, as would be understood by a person skilled in the art; a transgene encoding an RNA-guided RNA-targeting protein present downstream of the promoter, such that the transgene is expressed into its encoded transcripts and proteins within a mammalian cell; a polyA sequence downstream of said transgene that terminates transcription, as would be understood by a person having ordinary skill in the art. In one example, the RNA-guided RNA-targeting protein is a Type VI CRISPR-Cas. In another example, the Cas nuclease is a Cas13. In yet another example, the Cas nuclease is Cas13d from Ruminococcus flavefaciens (CasRX).

Also disclosed herein is a second promoter that drives the gRNA expression in a mammalian system (such as, but not limited to, the U6 or H1 promoters). Further disclosed herein is a sequence downstream of the second promoter, encoding the gRNA that complexes with the expressed CRISPR-Cas protein and guides it to a defined RNA sequence in the RNA viral genome. In one example, the one or more of guide RNA (gRNA)-encoding sequences, result in the expression of one or more guide RNAs (gRNAs) within the same cell. This allows for the targeting of multiple, unrelated sites within the viral genome in one expression construct. In another example, the guide RNAs (gRNAs) are as disclosed in Table 1.

In one example, the vector comprises two AAV2 inverted terminal repeats (ITRs) that allow for the expressed DNA construct to be packaged into adeno-associated virus (AAV) particles. When transduced into mammalian cells, the packaged AAV viruses allow for the expression of the Cas proteins and gRNAs.

Also disclosed herein is a method of determining gRNA efficiency, as well as a method of selecting gRNAs against viral RNA. In one example, the viral RNA is RNA from, but not limited to, the EV71, Coxsackievirus and Parechovirus (Echovirus) genomes. In another example, the methods disclosed herein are suitable for antiviral applications, such as, but not limited to, treatment of and prophylaxis against hand, foot, and mouth disease (HFMD). This is done by targeting the viral genome of, for example, but not limited to, Enterovirus 71. In one example, targets within the EV71 genome include, but is not limited to, the non-coding sequences that include the 5′UTRs and 3′UTRs, and the coding sequences of 2Apro, 2BC, 2B, 2C, 3AB, 3A, 3B (VPg), 3CDpro, 3Cpro, 3Dpol, VP1, VP2, VP3 and VP4. The Genbank Accession number for the referenced gene sequence EV-A71 is AF316321.2.

This disclosure describes technology and compositions for targeting and eliminating RNA viruses. The system disclosed herein utilises type VI CRISPR-Cas systems programmed to directly cleave the RNA viral genomes. Other types of CRISPR-Cas systems can be used in a similar manner. The composition and use here provide for the treatment and/or prevention of hand, foot, and mouth disease (HFMD); and/or reduction of Enterovirus 71 (EV71), Coxsackievirus (CAV16 and CAV6) and Parechovirus (Echovirus; Parechovirus A, Parechovirus B, Parechovirus C, Parechovirus D, Parechovirus E, and Parechovirus F), the viruses that cause hand, foot, and mouth disease (HFMD), in a subject, by cleavage of the RNA genome with the system disclosed herein. In one example, the system comprises CRISPR-Cas13. In another example, the Cas13d from Ruminococcus flavefaciens (CasRX). The type VI CRISPR-Cas systems target only the RNA viral genomes without risk of undesired activity against the host DNA genome.

The technology disclosed herein can be applied to any antiviral therapeutic application for RNA viruses by changing the guide sequences.

Thus, in one example, the target virus is an RNA virus. In one example, the RNA virus is a virus of the Picornaviridae family, which is a family of viruses comprising related non-enveloped RNA viruses which infect vertebrates, including mammals and birds. They are viruses that represent a large family of small, positive-sense, single-stranded RNA viruses with a 30-nm icosahedral capsid. The viruses of this family can cause a range of diseases including, but not limited to, the common cold, poliomyelitis, meningitis, hepatitis, and paralysis. Thus, in one example, the disease to be treated is, but is not limited to, polio; mild respiratory illness (the common cold); hand, foot, and mouth disease (HFMD); acute hemorrhagic conjunctivitis; aseptic meningitis; myocarditis; severe neonatal sepsis-like disease; acute flaccid paralysis; acute flaccid myelitis; Bornholm disease; epidemic pleurodynia; Herpangina; and chronic fatigue syndrome. In one example, the disease to be treated is hand, foot, and mouth disease (HFMD).

In another example, the RNA virus is a virus of the Enterovirus genus, which is a genus of positive-sense single-stranded RNA viruses associated with several human and mammalian diseases. Enteroviruses are named by their transmission-route through the intestine (enteric meaning intestinal). In another example, the molecular systems, methods of treatments, vectors, guide RNA molecules and CRISPR-Cas systems disclosed herein target at least one virus. The virus can be, but is not limited to Enterovirus, Coxsackie virus and Parechovirus. In another example, the virus can be, but is not limited to, Enterovirus 71, Coxsackie virus CAV16, Coxsackie virus CAV6, Parechovirus A, Parechovirus B, Parechovirus C, Parechovirus D, Parechovirus E, Parechovirus F, and combinations thereof.

Also contemplated in the present disclosure is the use of the described technology as a screening tool for comparative study of Cas enzyme proteins against RNA virus sequences.

The CRISPR-Cas therapeutics offer a new class of anti-viral therapeutics that can be easily manufactured on a large scale basis for distribution. The results shown herein show that by targeting, for example, EV71 nucleic acid using crRNA, the RNA-targeting RNase CasRX would be able to significantly inhibit the replication of the viruses. This is a demonstration of AAV-packaged anti-viral CRISPR-Cas tools that are shown to work effectively against RNA viruses, based on the example of EV71, as shown in the data presented herewith. This approach allows delivery of therapeutics into an in vivo system, such as a mammalian or human body, for targeting the virus replication in the virus-infected cells throughout the body. In the selection of the guide molecules disclosed herein, the crRNA guides have been selected to avoid any possible off-target excisions to the human genome.

The data presented herewith also shows that this antiviral approach is dependent on the MOI of AAV-therapeutics that were used for the transduction, as well as the dynamics of the pooled crRNA guides. This, a pool of six crRNA guides has been identified as a cocktail for the effective inhibition of the EV71 virus replication.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Design and synthesis of CasRX construct. The CasRX sequence was obtained from Addgene (pXR001: EF1a-CasRX-2A-EGFP, plasmid no. 109049). The CasRX was cloned with a HA tag and a rabbit polyA tail (CasRX-HA-polyA) driven under a mammalian CMV promoter. Immediately downstream of the expression cassette, the gRNA backbone driven by human U6 promoter was cloned in and the sequence was obtained from Addgene (pXR003: CasRX gRNA cloning backbone, plasmid no. 109053).

Cell lines and viruses. Human rhabydosarcoma (RD) cells were purchased from the American Type Culture Collection (ATCC). Human B4G12 cells were purchased from Creative Bioarray. The cells were grown in media recommended by the ATCC and Creative Bioarray respectively. AAVs were generated in-house. Briefly, AAV were packaged via a triple transfection of 293AAV cell line (Cell Biolabs AAV-100) that were plated in a HYPERFlask ‘M’ (Corning) in growth media consisting of DMEM+glutaMax+pyruvate+10% FBS (Thermo Fisher), supplemented with 1×MEM non-essential amino acids (Gibco). Confluency at transfection was between 70 to 90%. Media was replaced with fresh pre-warmed growth media before transfection. For each HYPERFlask ‘M’, 200 μg of pHelper (Cell Biolabs), 100 μg of pRepCap [encoding capsid proteins for serotype DJ or 2], and 100 μg of pZac-CASI-GFP or pZac-CMV-CasRX were mixed in 5 ml of DMEM, and 2 mg of PEI “MAX” (Polysciences) (40 kDa, 1 mg/ml in H₂O. pH 7.1) added for PEI:DNA mass ratio of 5:1. The mixture was incubated for 15 minutes, and transferred drop-wise to the cell media. The day after transfection, media was changed to DMEM+glutamax+pyruvate+2% FBS. Cells were harvested 48 to 72 hours after transfection by scrapping or dissociation with 1×PBS (pH7.2)+5 mM EDTA, and pelleted at 1500 g for 12 minutes. Cell pellets were re-suspended in 1-5 ml of lysis buffer (Tris HCl PH 7.5, 2 mM MgCl, 150 mM NaCl), and freeze-thawed 3 times between dry-ice-ethanol bath and 37° C. water bath. Cell debris was clarified via 4000 g for 5 minutes, and the supernatant collected. The collected supernatant was treated with 50 U/ml of Benzonase (Sigma-Aldrich) and 1 U/ml of RNase cocktail (Invitrogen) for 30 min at 37° C. to remove unpackaged nucleic acids. After incubation, the lysate was loaded on top of a discontinuous density gradient consisting of 6 ml each of 15%, 25%, 40%, 60% Optiprep (Sigma-Aldrich) in an 29.9 ml Optiseal polypropylene tube (Beckman-Coulter). The tubes were ultra-centrifuged at 54000 rpm, at 18° C., for 1.5 hours, on a Type 70 Ti rotor. The 40% fraction was extracted, and dialyzed with 1×PBS (pH 7.2) supplemented with 35 mM NaCl, using Amicon Ultra-15 (100 kDa MWCO) (Millipore). The titer of the purified AAV vector stocks were determined using real-time qPCR with ITR-sequence-specific primers and probe, referenced against the ATCC reference standard material 8 (ATCC). The Enterovirus strain used is EV-A71 strain 5865/sin/000009.

Immunofluorescence assay. 10,000 human immortalized corneal endothelial cells, B4G12, were plated on glass slides in a 48-well plate and transduced with AAVDJ-CasRX or AAVDJ-GFP at MOI 100K. At 3 days post-transduction, cells were fixed and permeabilized using methanol for 10 mins and blocked using 1×PBS with 5% BSA for 1 hour. This is followed by primary antibody anti-HA (abcam) incubation at 1:200 dilution for 2 hours at room temperature or anti-GFP (abcam) at 1:1500 for 2 hours at room temperature. Secondary antibodies staining is carried out at 1:1000 (Thermo Fisher) for 2 hours at room temperature. Slides were then washed 3 times with 1×PBS and mounted on to the slide using ProLong mounting medium (Thermo). Images are taken using an Olympus microscope, exposure set at 15 milliseconds and gain set at 6400 for bright field and the 488 channel, exposure is set at 800 us and gain set at 6400 for the DAPI channel.

Biopanning assay for selection of AAV serotype. Immortalized human rhabdomyosarcoma (RD) cells were seeded in a 48-well plate at a density of 10,000 cells per well in 200 μl DMEM containing 10% FBS. The cells were cultured overnight at 37° C. and allowed to adhere to the well. A panel of AAV (1, 2, 6, 7, 8, 9, rh10, DJ and Anc80) were used to transduce the cells at MOI of 100K, in triplicates. At 72 hours post-transduction, the cells were harvested and the total GFP protein is quantitated using a GFP quantification kit (Biovision) on a multi-well plate reader (Tecan).

RNA gene interference activity in AAVDJ-CasRX transduced cells. Immortalized human rhabdomyosarcoma (RD) cells were seeded in a 48-well plate at a density of 10,000 cells per well in 200 μl DMEM containing 10% FBS. The cells were transduced with AAV2-GFP at MOI of 10K for expression of GFP. The GFP knockdown efficiency of guide1 only, guide2 only and guide1+guide2 are tested by transduction of the GFP-expressing rhabdomyosarcoma (RD) cells with AAV2-CasRX bearing the respective guides at of MOI 100K and MOI 1000K. At 72 hours post-transduction, the cells were harvested and the total GFP protein is quantitated using a GFP quantification kit (Biovision) on a multi-well plate reader (Tecan).

In vitro antiviral plaque assay with EV71. For screening of anti-EV71 activity, rhabdomyosarcoma (RD) cells were seeded in 96-well plates at a density of 10⁴ cells per well and incubated overnight in an incubator. Perform dilution of AAV2-CasRX bearing the different guides for transduction individually or in pooled format at MOI 1K, 10K, 100K and 1000K in a 100 ul volume. At 72 hours post-transduction, the cells were infected with EV-A71 virus at MOI of 1. The plate was washed twice with 1×PBS and incubated with DMEM with 2% FBS for 12 h. The supernatant from each well were harvested at 12 hours post-infection and used for subsequent virus plaque assay. For virus plaque assay, rhabdomyosarcoma (RD) cells were seeded in 24-well plates and incubated with 10 to 10⁶ fold serially diluted supernatant samples in a 100 ul volume. Plates were washed twice with PBS and overlay media were added to each well and incubated with 15 mins rocking interval for an hour before leaving it in the incubator for 4 days. After 4 days of incubation, the overlay media was removed and crystal violet stain was added to visualize the plaques for counting.

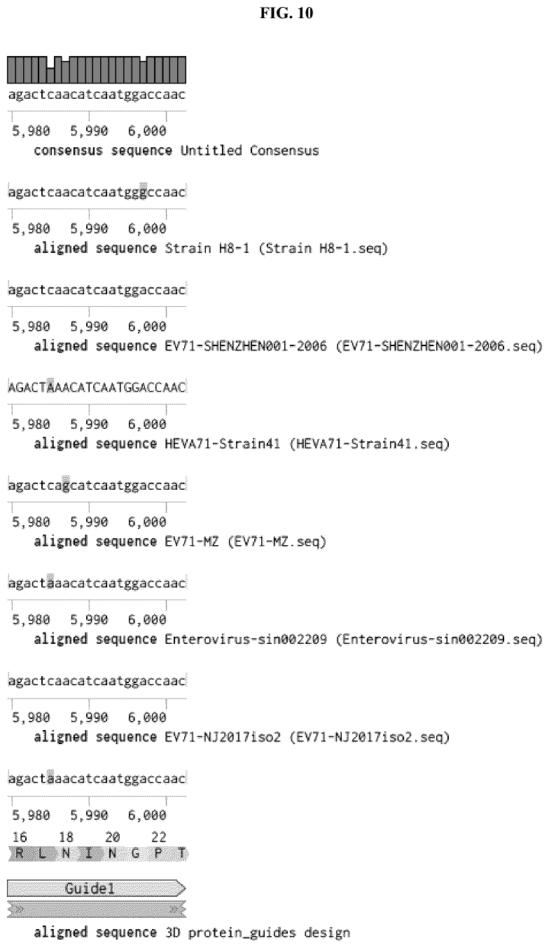

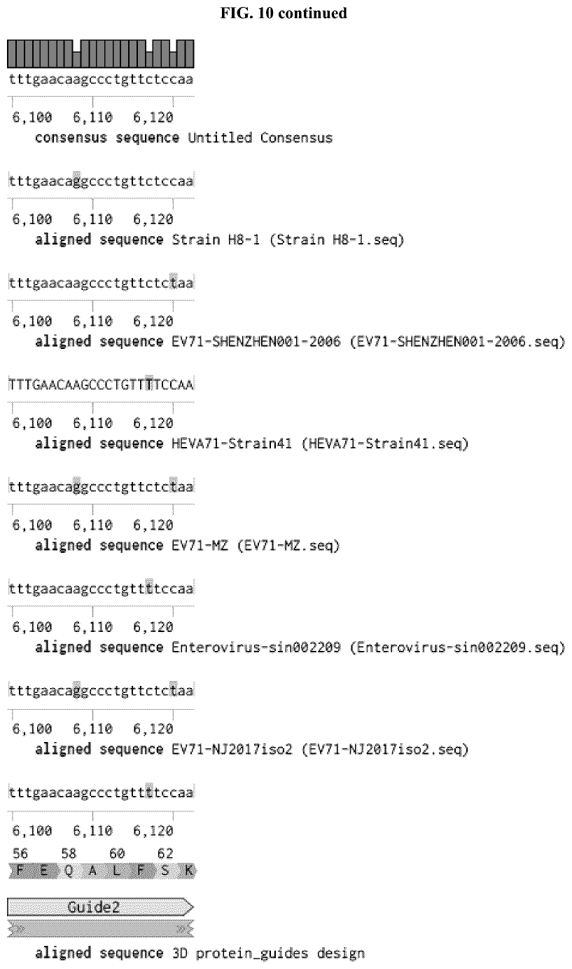

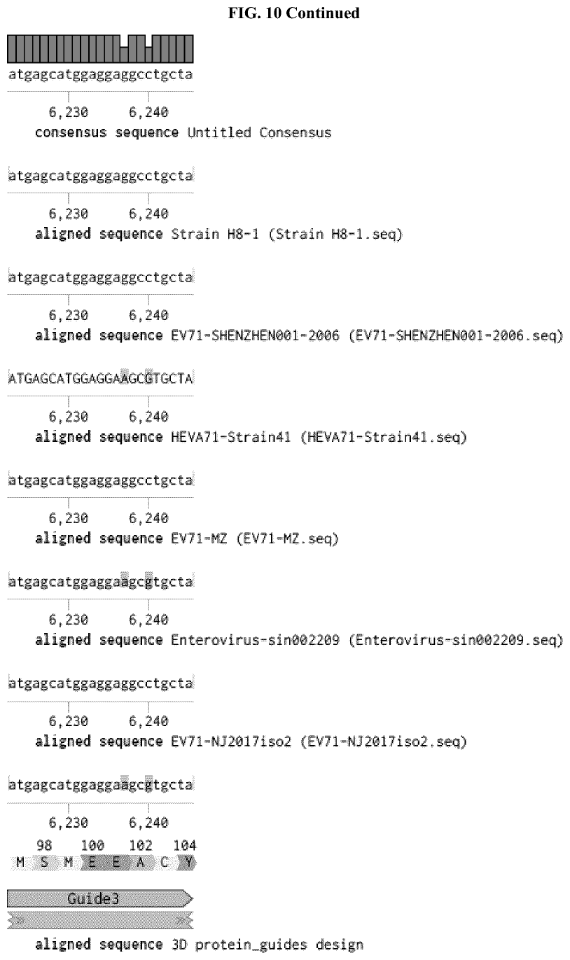

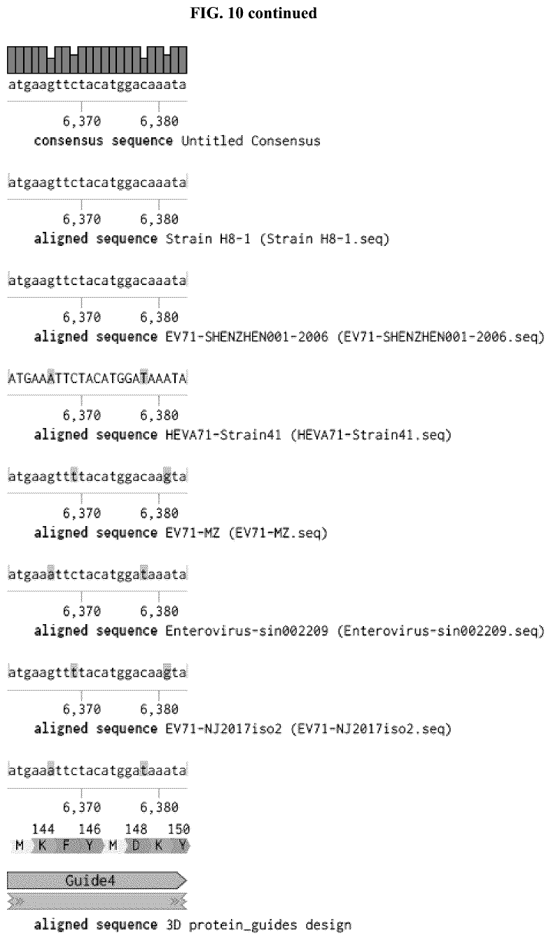

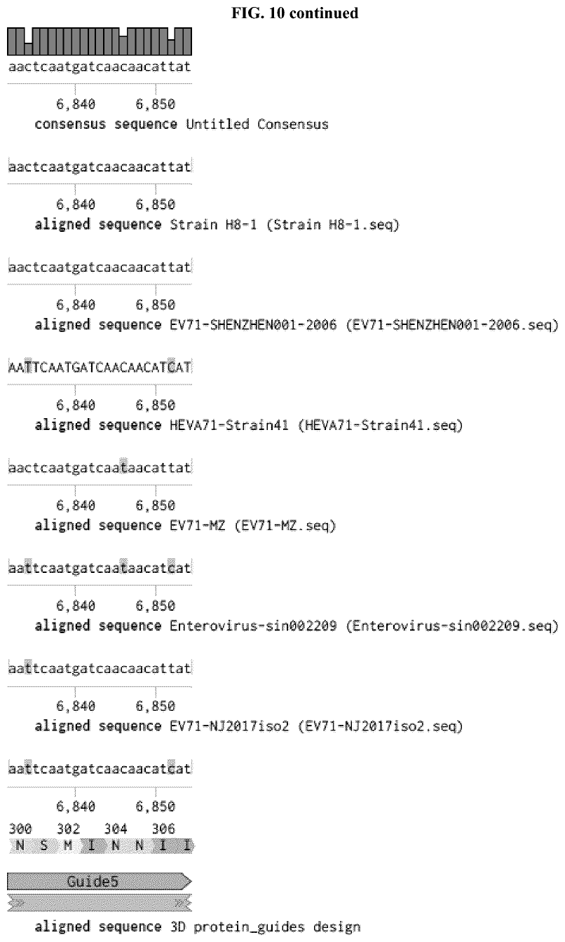

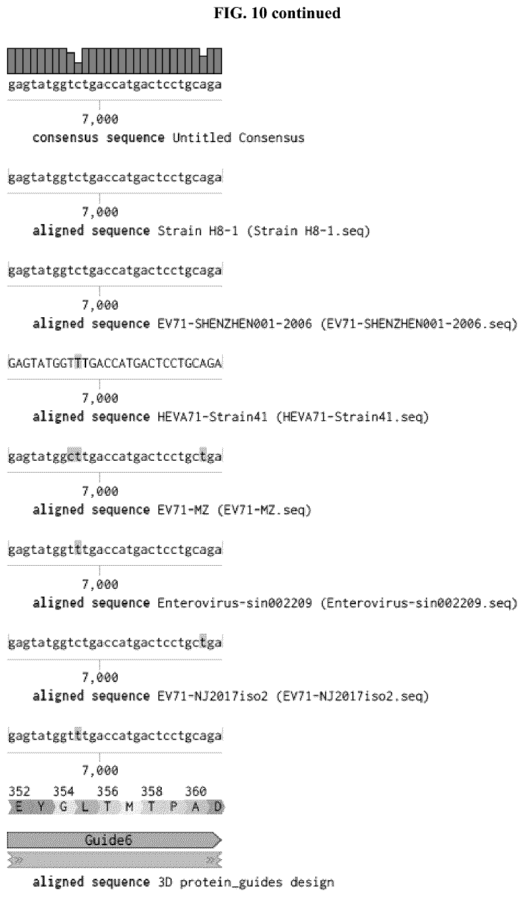

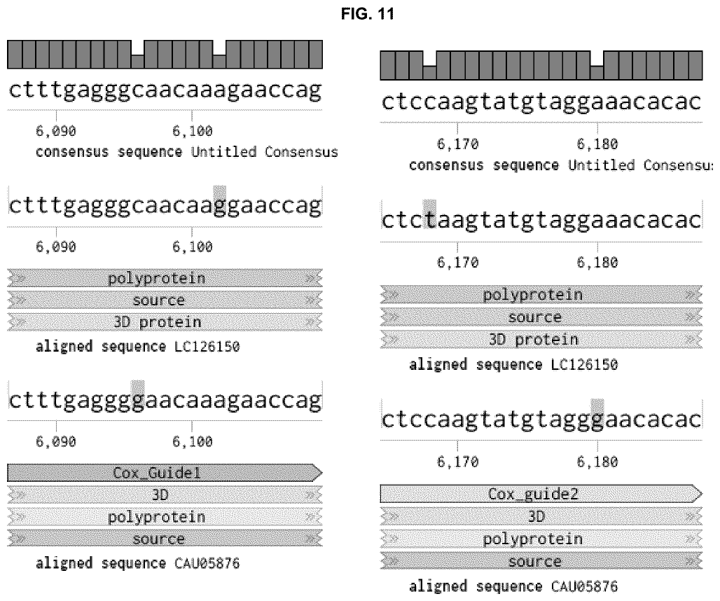

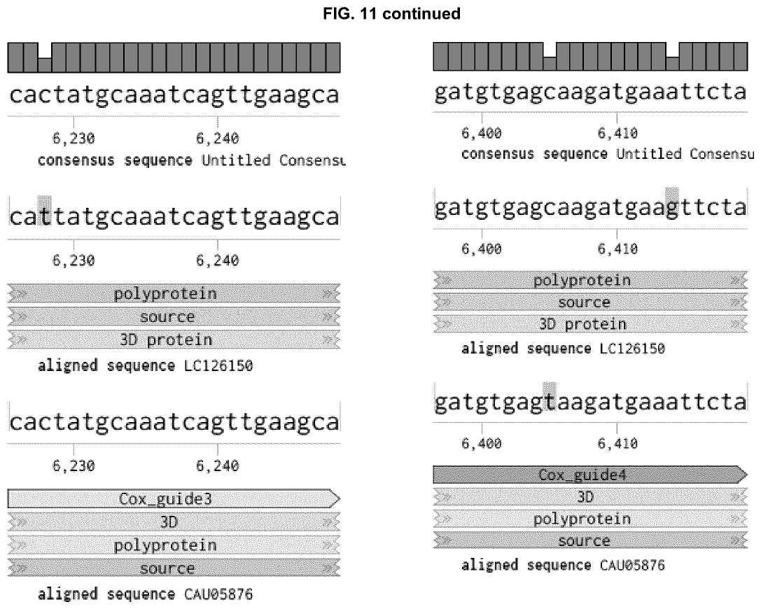

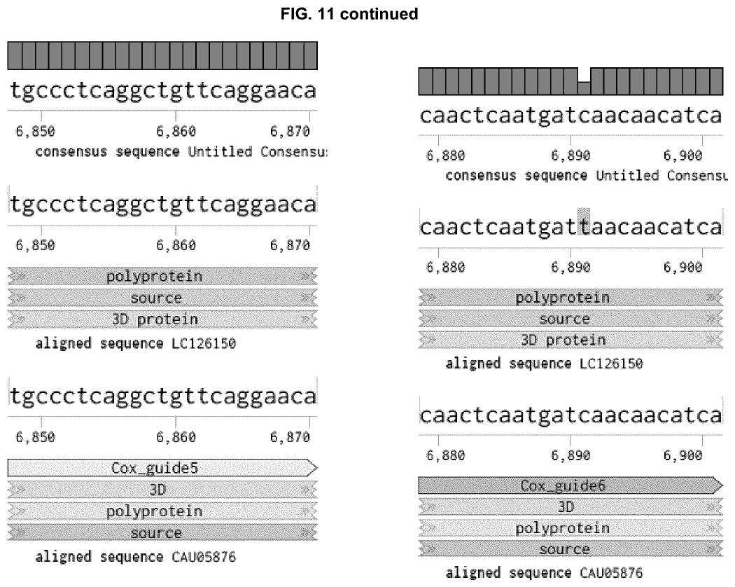

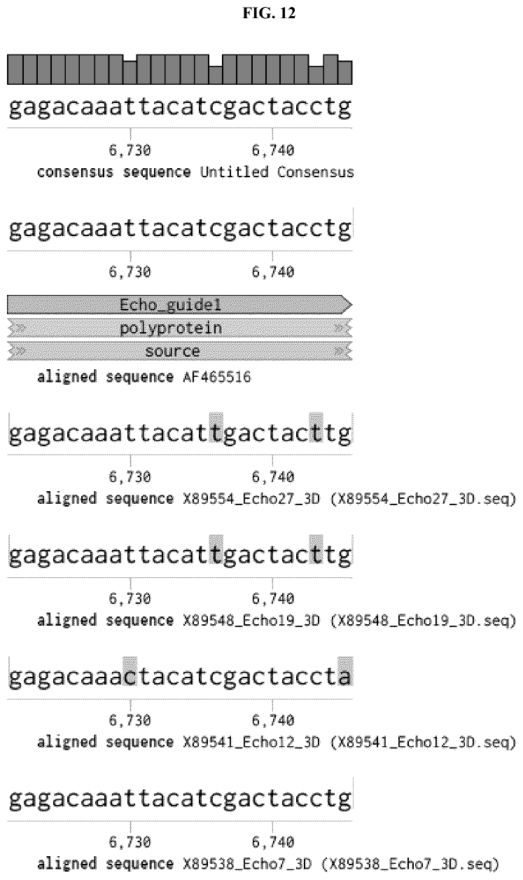

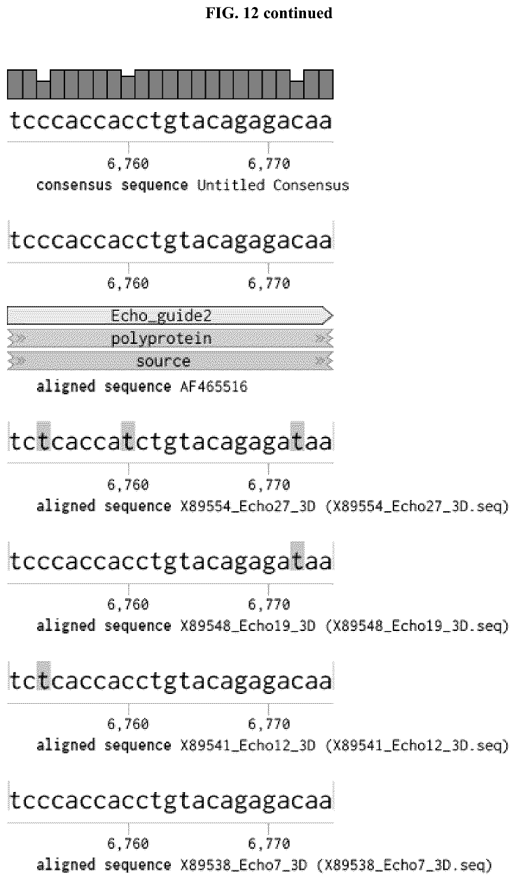

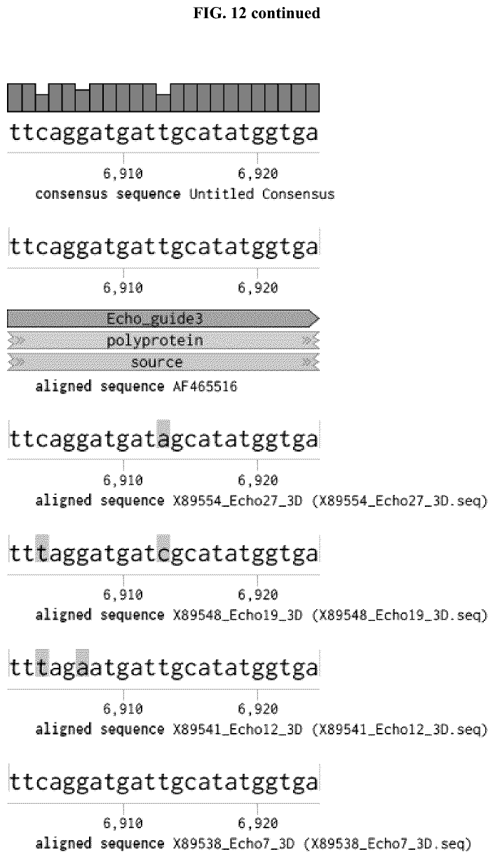

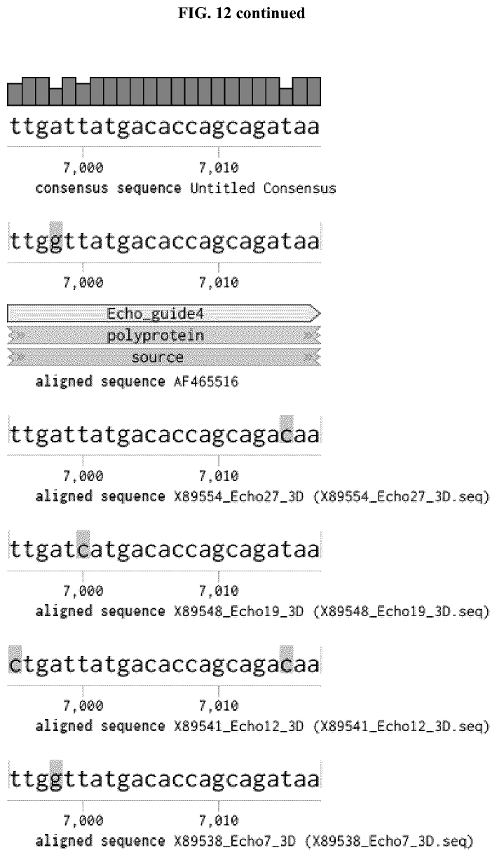

TABLE 1
TABLE OF SEQUENCES

| SEQ | | |
|---|---|---|
| ID | | |
| NO: | Comments/Annotations | Sequence |
| 1 | EV71_3D guide1 sequence | gttggtccattgatgtttagtct |
| 2 | EV71_3D guide2 sequence | ttggaaaacagggcttgttcaaa |
| 3 | EV71_3D guide3 sequence | tagcacgcttcctccatgctcat |
| 4 | EV71_3D guide4 sequence | tatttatccatgtagaatttcat |
| 5 | EV71_3D guide5 sequence | atgatgttgttgatcattgaatt |
| 6 | EV71_3D guide6 sequence | tctgcaggagtcatggtcaaaccatactc |
| 7 | GFP guide1 sequence | gtgaacagctcctcgcccttgct |
| 8 | GFP guide2 sequence | gctgaacttgtggccgtttac |
| 9 | GG001; Primer for cloning pZac- | ATGGTCTCCATCTGCTTATATAGACCTCCCAC |
| | CMV-CasRx-polyA-U6-gRNA | CGT |
| 10 | GG002; Primer for cloning pZac- | ATGGTCTCAAGATGGGCGTGAAGTCCACACT |
| | CMV-CasRx-polyA-U6-gRNA | |
| 11 | GG003; Primer for cloning pZac- | ATGGTCTCCTATCCGGATCCGGAATTGCCG |
| | CMV-CasRx-polyA-U6-gRNA | |
| 12 | GG004; Primer for cloning pZac- | ATGGTCTCGGATACCCATACGATGTTCCAGAT |
| | CMV-CasRx-polyA-U6-gRNA | TACGCT |
| 13 | GG005; Primer for cloning pZac- | TAGGTCTCACATTAGGCATAGTCGGGGACAT |
| | CMV-CasRx-polyA-U6-gRNA | CATATG |
| 14 | GG006; Primer for cloning pZac- | ATGGTCTCCAATGGCTAATAAAGGAAATTTAT |
| | CMV-CasRx-polyA-U6-gRNA | TTTCATTGCAATAGTGTG |
| 15 | GG007; Primer for cloning pZac- | ATGGTCTCACTCGATCTCCATAAGAGAAGAG |
| | CMV-CasRx-polyA-U6-gRNA | GGACAGCT |
| 16 | GG008; Primer for cloning pZac- | ATGGTCTCCCGAGGGCCTATTTCCCATGATTC |
| | CMV-CasRx-polyA-U6-gRNA | CTTC |
| 17 | GG009; Primer for cloning pZac- | ATGGTCTCAATTCACGACACCTGAAATGGAAG |
| | CMV-CasRx-polyA-U6-gRNA | AAA |
| 18 | GG0010; Primer for cloning | ATGGTCTCGGAATGGGCGTGAAGTCCACACT |
| | pZac-CMV-CasRx-polyA-U6- | |
| | gRNA | |
| 19 | EK19001; Primer for cloning | CCTACCAACTGGTCGGGGTTTGGTTGGTCCA |
| | EV71_3D_guide1 into pZac | TTGATGTTTAGTCT |
| | backbone | |
| 20 | EK19002; Primer for cloning | GGAGAAAATACCGCATCAGAATTCAAAAGACT |
| | EV71_3D_guide1 into pZac | AAACATCAATGGACCAAC |
| | backbone | |
| 21 | EK19003; Primer for cloning | CCTACCAACTGGTCGGGGTTTGTTGGAAAAC |
| | EV71_3D_guide2 into pZac | AGGGCTTGTTCAAA |
| | backbone | |
| 22 | EK19004; Primer for cloning | GGAGAAAATACCGCATCAGAATTCAAATTTGA |
| | EV71_3D_guide2 into pZac | ACAAGCCCTGTTTTCCAA |
| | backbone | |
| 23 | EK19005; Primer for cloning | CCTACCAACTGGTCGGGGTTTGTAGCACGCT |
| | EV71_3D_guide3 into pZac | TCCTCCATGCTCAT |
| | backbone | |
| 24 | EK19006; Primer for cloning | GGAGAAAATACCGCATCAGAATTCAAAATGAG |
| | EV71_3D_guide3 into pZac | CATGGAGGAAGCGTGCTA |
| | backbone | |
| 25 | EK19007; Primer for cloning | CCTACCAACTGGTCGGGGTTTGTATTTATCCA |
| | EV71_3D_guide4 into pZac | TGTAGAATTTCAT |
| | backbone | |
| 26 | EK19008; Primer for cloning | GGAGAAAATACCGCATCAGAATTCAAAATGAA |
| | EV71_3D_guide4 into pZac | ATTCTACATGGATAAATA |
| | backbone | |
| 27 | EK19009; Primer for cloning | CCTACCAACTGGTCGGGGTTTGATGATGTTGT |
| | EV71_3D_guide5 into pZac | TGATCATTGAATT |
| | backbone | |
| 28 | EK19010; Primer for cloning | GGAGAAAATACCGCATCAGAATTCAAAAATTC |
| | EV71_3D_guide5 into pZac | AATGATCAACAACATCAT |
| | backbone | |
| 29 | EK19011; Primer for cloning | CCTACCAACTGGTCGGGGTTTGTCTGCAGGA |
| | EV71_3D_guide6 into pZac | GTCATGGTCAAACCATACTC |
| | backbone | |
| 30 | EK19012; Primer for cloning | GGAGAAAATACCGCATCAGAATTCAAAGAGTA |
| | EV71_3D_guide6 into pZac | TGGTTTGACCATGACTCCTGCAGA |
| | backbone | |
| 31 | SV038; primer for cloning | AACTGGTCGGGGTTTGGTGAACAGCTCCTCG |
| | GFP_guide1 into pZac | CC |
| | backbone | |
| 32 | SV039; primer for cloning | GAAAATACCGCATCAGAATTCAAAAAAAAGCA |
| | GFP_guide1 into pZac | AGGGCGAGGAGC |
| | backbone | |
| 33 | SV040; primer for cloning | AACTGGTCGGGGTTTGGCTGAACTTGTGGCC |
| | GFP_guide2 into pZac | GTT |
| | backbone | |
| 34 | SV041; primer for cloning | CCGCATCAGAATTCAAAAAAAGTAAACGGCCA |
| | GFP_guide2 into pZac | CAAGTTCAG |
| | backbone | |
| 35 | 3D_protein_guides_desig 1395 | atgcaaggtgagatccaatgggtgaagcctaacaaggaaactgg |
| | bp ds-DNA | cagactaaacatcaatggaccaactcgcactaagttggagcctag |
| | FEATURES: Location/Qualifiers | tgtatttcatgatgtgtttgaaggcaacaaggaaccagcagttttaac |
| | CDS: 1 . . . 1392 | aagtaaagaccctagattggaggtcgactttgaacaagccctgtttt |
| | mat_peptide: 7 . . . 1392 | ccaagtatgtgggcaatgttttacacgagcccgatgaatatgtgact |
| | /label=“Geneious name: 3D | caagctgccctccactatgcgaatcaacttaaacaattggacataa |
| | peptide″ | acactagcaagatgagcatggaggaagcgtgctatggcactgaa |
| | misc_feature: 46 . . . 68 | aacctggaagcaatagacctctgcactagtgctgggtatccataca |
| | /label=“Guide1″ | gtgcccttggtatcaagaaaagagacattctcgaccccataacca |
| | misc_feature: 166 . . . 188 | gggatgtgtctaagatgaaattctacatggataaatacggactaga |
| | /label=“Guide2″ | tctgccatactctacctatgtgaaggatgaacttagatctctggataa |
| | misc_feature: 289 . . . 311 | aatcaagaaaggaaagtcacgcctgatagaggccagcagcttg |
| | /label=“Guide3″ | aatgactctgtctacctcagaatgacttttgggcacctttacgaggtgt |
| | misc_feature: 427 . . . 449 | ttcatgctaaccctggtactgtgactggctcagcagtaggttgcaac |
| | /label=“Guide4″ | ccagacgtgttttggagtaaactaccgattctgctgcctgggtcactc |
| | misc_feature: 898 . . . 920 | tttgcctttgactactcaggatatgatgctagtctcagcccggtatggtt |
| | /label=“Guide5″ | cagggctctagaagttgtgttacgggagattgggtattcagaggag |
| | misc_feature: 1054 . . . 1082 | gccgtgtccctaatagaaggaatcaaccacacccaccatgtgtac |
| | /label=“Guide6″ | cggaataaaacatactgtgtacttggtgggatgccctcagggtgct |
| | | ctggtacttccatcttcaattcaatgatcaacaacatcatcattagaa |
| | | cccttttgatcaaaacctttaagggaatagacctggatgagttgaac |
| | | atggtggcctatggggacgatgtgctggccagttacccttttcctattg |
| | | attgccttgaattggctaagactggcaaagagtatggtttgaccatg |
| | | actcctgcagacaaatcaccctgtttcaatgaagtaacatgggaga |
| | | atgctaccttcctgaagagagggttcttgccagaccaccaatttcca |
| | | ttcttaattcaccctacgatgcccatgagagagatccatgagtccatt |
| | | cgatggactaaggacgcgcgtaacacccaggatcacgtgcgctc |
| | | cctgtgtctattggcatggcacaatggtaaggatgaatatgaaaagt |
| | | ttgtgagtgcaattagatcagttccagttggaaaagcgttggccattc |
| | | ctaactttgagaatctgagaagaaattggctcgaattgttttaa |
| 36 | GFP_guides_design 746 bp ds- | Atggtgagcaagggcgaggagctgttcaccggggtggtgcccat |
| | DNA circular | cctggtcgagctggacggcgacgtaaacggccacaagttcagcg |
| | FEATURES: Location/Qualifiers | tgtccggcgagggcgagggcgatgccacctacggcaagctgac |
| | CDS: 1 . . . 746/label=“eGFP″ | cctgaagttcatctgcaccaccggcaagctgcccgtgccctggcc |
| | CDS: 1 . . . 746/label=“Translation | caccctcgtgaccaccctgacctacggcgtgcagtgcttcagccg |
| | 1-746″ | ctaccccgaccacatgaagcagcacgacttcttcaagtccgccat |
| | misc_feature: 7 . . . 29 | gcccgaaggctacgtccaggagcgcaccatcttcttcaaggacg |
| | /label=“Knockdown_Guide1″ | acggcaactacaagacccgcgccgaggtgaagttcgagggcga |
| | misc_feature: 67 . . . 87 | caccctggtgaaccgcatcgagctgaagggcatcgacttcaagg |
| | /label=“Knockdown_Guide2″ | aggacggcaacatcctggggcacaagctggagtacaactacaa |
| | | cagccacaacgtctatatcatggccgacaagcagaagaacggc |
| | | atcaaggtgaacttcaagatccgccacaacatcgaggacggcag |
| | | cgtgcagctcgccgaccactaccagcagaacacccccateggc |
| | | gacggccccgtgctgctgcccgacaaccactacctgagcaccca |
| | | gtccgccctgagcaaagaccccaacgagaagcgcgatcacatg |
| | | gtcctgctggagttcgtgaccgccgccgggatcactctcggcatgg |
| | | acgagctgtacaagtaaATCACGACgacgagctgtacaagt |
| | | aa |
| 37 | See end of table | See end of table |
| 38 | primer complement(121 . . . 144) | GGGAGAGGCGGTTTGCGTATTGGG |
| | /label=“2225″of SEQ ID NO: 37 | |
| 39 | primer 534 . . . 557 | gccatgctctaggaagatcgggac |
| | /label=“EK18011” of SEQ ID | |
| | NO: 37 | |
| 40 | primer 534 . . . 593 | gccatgctctaggaagatcgggacattgattattgactagttattaat |
| | /label=“EK18001” of SEQ ID | agtaatcaatta |
| | NO: 37 | |
| 41 | primer 622 . . . 645 /label=“1075” of | CGGAGTTCCGCGTTACATAACTTAC |
| | SEQ ID NO: 37 | |
| 42 | primer 722 . . . 741/label=“1324” of | ACGCCAATAGGGACTTTCCA |
| | SEQ ID NO: 37 | |
| 43 | primer 811 . . . 830/label=“2394” of | AAGTACGCCCCCTATTGACG |
| | SEQ ID NO: 37 | |
| 44 | primer complement(1052 . . . 1077) | gagttgttacgacattttggaaagtc |
| | /label=“EK18015” of SEQ ID | |
| | NO: 37 | |
| 45 | primer | ATGGTCTCCatctgcttatatagacctcccaccgt |
| | complement(1111 . . . 1134) | |
| | /label=“GG001” of SEQ ID NO: | |
| | 37 | |
| 46 | primer complement(1111 . . . 1134) | tgtggacttcacgcccatggtggcctgcttatatagacctcccaccgt |
| | /label=“SV036” of SEQ ID NO: | |
| | 37 | |
| 47 | primer | ATGGTCTCGgatacccatacgatgttccagattacgct |
| | 4018 . . . 4044/label=“GG004” of | |
| | SEQ ID NO: 37 | |
| 48 | primer | TAGGTCTCAcattaggcatagtcggggacatcatatg |
| | complement(4076 . . . 4100)/label=“ | |
| | GG005″of SEQ ID NO: 37 | |
| 49 | primer 4102 . . . 4140 | ATGGTCTCCaatggctaataaaggaaatttattttcattgcaat |
| | /label=“GG006” of SEQ ID NO: | agtgtg |
| | 37 | |
| 50 | primer complement(4462 . . . 4488) | ATGGTCTCActcgatctccataagagaagagggacagct |
| | /label=“GG007″of SEQ ID NO: | |
| | 37 | |
| 51 | primer 4489 . . . 4510 | gagggcctatttcccatgattc |
| | /label=“U6_fwd_seqchk” of SEQ | |
| | ID NO: 37 | |
| 52 | primer 4489 . . . 4514 | ATGGTCTCCcgagggcctatttcccatgattccttc |
| | /label=“GG008” of SEQ ID NO: | |
| | 37 | |
| 53 | Primer complement(4490 . . . 4511) | ggaatcatgggaaataggccct |
| | /label=“13 EF1a_R (out)” of | |
| | SEQ ID NO: 37 | |
| 54 | primer complement(4713 . . . 4738) | CGGTGTTTCGTCCTTTCCACAAGATA |
| | /label=“736” of SEQ ID NO: 37 | |
| 55 | primer 4743 . . . 4759 | cctaccaactggtcggg |
| | /label=“EK_GS_F1” of SEQ ID | |
| | NO: 37 | |
| 56 | primer 4743 . . . 4764 | cctaccaactggtcggggtttg |
| | /label=“EK18100” of SEQ ID | |
| | NO: 37 | |
| 57 | primer complement(4791 . . . 4817) | GGAGAAAATACCGCATCAGAATTCAAA |
| | /label=“EK18101” of SEQ ID | |
| | NO: 37 | |
| 58 | primer complement(4796 . . . 4817) | ggagaaaataccgcatcagaat |
| | /label=“EK_GS_R1” of SEQ ID | |
| | NO: 37 | |
| 59 | primer complement(4843 . . . 4895) | gctacttatctacgtagccatgcgaattactatggttgctttgacgtat |
| | /label=“EK18008” of SEQ ID | gcgg |
| | NO: 37 | |
| 60 | primer complement(4866 . . . 4895) | gctacttatctacgtagccatgcgaattac |
| | /label=“EK18012” of SEQ ID | |
| | NO: 37 | |
| 61 | primer complement(5206 . . . 5225) | TTCAGGCTGCGCAACTGTTG |
| | /label=“2452” of SEQ ID NO: 37 | |
| 62 | primer 5824 . . . 5849 | ATGAGTATTCAACATTTCCGTGTCGC |
| | /label=“1625” of SEQ ID NO: 37 | |
| 63 | primer complement(7226 . . . 7246) | GCTCACGCTGTAGGTATCTCA |
| | /label=“2372” of SEQ ID NO: 37 | |
| 64 | consensus sequence-untitled | agactcaacatcaatggaccaac |
| | consensus; and aligned | |
| | sequences-EV71- | |
| | SHENZEN001-2006; and EV71- | |
| | NJ2017iso2 of FIG. 10 | |
| 65 | aligned sequence-Strain H8-1 | agactcaacatcaatgggccaac |
| | of FIG. 10 | |
| 66 | aligned sequences-HEVA71- | agactaaacatcaatggaccaac |
| | Strain41, Enterovirus- | |
| | sin002209, and nucleic acid | |
| | sequence of Guide1 of FIG. 10 | |
| 67 | aligned sequence-EV71 MZ of | agactcagcatcaatggaccaac |
| | FIG. 10 | |
| 68 | Guide1 protein sequence of | RLNINGPT |
| | FIG. 10 | |
| 69 | consensus sequence-untitled | tttgaacaagccctgttctccaa |
| | consensus of FIG. 10 | |
| 70 | aligned sequence-Strain H8-1 | tttgaacaggccctgttctccaa |
| | of FIG. 10 | |
| 71 | aligned sequence-EV71- | tttgaacaagccctgttctctaa |
| | SHENZEN001-2006 of FIG. 10 | |
| 72 | aligned sequences-HEVA71- | tttgaacaagccctgttttccaa |
| | Strain41, Enterovirus- | |
| | sin002209, and nucleic acid | |
| | sequence of Guide2 of FIG. 10 | |
| 73 | aligned sequences-EV71 MZ, | tttgaacaggccctgttctctaa |
| | and EV71-NJ2017iso2 of FIG. | |
| | 10 | |
| 74 | Guide2 protein sequence of | FEQALFSK |
| | FIG. 10 | |
| 75 | consensus sequence-untitled | atgagcatggaggaggcctgcta |
| | consensus; and aligned | |
| | sequences-Strain H8-1, EV71- | |
| | SHENZEN001-2006, EV71-MZ, | |
| | and EV71-NJ2017iso2 of FIG. | |
| | 10 | |
| 76 | aligned sequences-HEVA71- | atgagcatggaggaagcgtgcta |
| | Strain41, Enterovirus- | |
| | sin002209, and nucleic acid | |
| | sequence of Guide3 of FIG. 10 | |
| 77 | Guide3 protein sequence of | MSMEEACY |
| | FIG. 10 | |
| 78 | consensus sequence-untitled | atgaagttctacatggacaaata |
| | consensus; and aligned | |
| | sequences-Strain H8-1, and | |
| | EV71-SHENZEN001-2006 of | |
| | FIG. 10 | |
| 79 | aligned sequences-HEVA71- | atgaaattctacatggataaata |
| | Strain41, Enterovirus- | |
| | sin002209, and nucleic acid | |
| | sequence of Guide4 of FIG. 10 | |
| 80 | aligned sequences-EV71-MZ | atgaagttttacatggacaagta |
| | and EV71-NJ2017iso2 of FIG. | |
| | 10 | |
| 81 | Guide4 protein sequence of | MKFYMDKY |
| | FIG. 10 | |
| 82 | consensus sequence-untitled | aactcaatgatcaacaacattat |
| | consensus; and aligned | |
| | sequences-Strain H8-1 and | |
| | EV71-SHENZEN001-2006 of | |
| | FIG. 10 | |
| 83 | aligned sequence-HEVA71- | aattcaatgatcaacaacatcat |
| | Strain41 and nucleic acid | |
| | sequence of Guide5 of FIG. 10 | |
| 84 | aligned sequence-EV71-MZ of | aactcaatgatcaataacattat |
| | FIG. 10. | |
| 85 | aligned sequence-Enterovirus- | aattcaatgatcaataacatcat |
| | sin002209 of FIG. 10 | |
| 86 | aligned sequence-EV71- | aattcaatgatcaacaacattat |
| | NJ2017iso2 of FIG. 10 | |
| 87 | Guide5 protein sequence of | NSMINNII |
| | FIG. 10 | |
| 88 | consensus sequence-untitled | gagtatggtctgaccatgactcctgcaga |
| | consensus; and aligned | |
| | sequences-Strain H8-1 and | |
| | EV71-SHENZEN001-2006 of | |
| | FIG. 10 | |
| 89 | aligned sequence-HEVA71- | gagtatggtttgaccatgactcctgcaga |
| | Strain41, Enterovirus- | |
| | sin002209, and nucleic acid | |
| | sequence of Guide6 of FIG. 10 | |
| 90 | aligned sequence-EV71-MZ of | gagtatggcttgaccatgactcctgctga |
| | FIG. 10. | |
| 91 | aligned sequence-EV71- | gagtatggtctgaccatgactcctgctga |
| | NJ2017iso2 of FIG. 10 | |
| 92 | Guide6 protein sequence of | EYGLTMTPAD |
| | FIG. 10 | |
| 93 | consensus sequence-untitled | ctttgagggcaacaaagaaccag |
| | consensus of FIG. 11 | |
| 94 | aligned sequence-LC126150 of | ctttgagggcaacaaggaaccag |
| | FIG. 11 | |
| 95 | aligned sequence-CAU05876; | ctttgaggggaacaaagaaccag |
| | Cox_Guide1 of FIG. 11 | |
| 96 | consensus sequence-untitled | ctccaagtatgtaggaaacacac |
| | consensus of FIG. 11 | |
| 97 | aligned sequence-LC126150 of | ctctaagtatgtaggaaacacac |
| | FIG. 11 | |
| 98 | aligned sequence-CAU05876; | ctccaagtatgtagggaacacac |
| | Cox_Guide2 of FIG. 11 | |
| 99 | consensus sequence-untitled | cactatgcaaatcagttgaagca |
| | consensus of FIG. 11; aligned | |
| | sequence-CAU05876; | |
| | Cox_Guide3 of FIG. 11 | |
| 100 | aligned sequence-LC126150 of | cattatgcaaatcagttgaagca |
| | FIG. 11 | |
| 101 | consensus sequence-untitled | gatgtgagcaagatgaaattcta |
| | consensus of FIG. 11 | |
| 102 | aligned sequence-LC126150 of | gatgtgagcaagatgaagttcta |
| | FIG. 11 | |
| 103 | aligned sequence-CAU05876; | gatgtgagtaagatgaaattcta |
| | Cox_Guide4 of FIG. 11 | |
| 104 | consensus sequence-untitled | tgccctcaggctgttcaggaaca |
| | consensus; aligned sequences- | |
| | LC126150 and CAU05876; | |
| | Cox_Guide5 of FIG. 11 | |
| 105 | consensus sequence-untitled | caactcaatgatcaacaacatca |
| | consensus; aligned sequence- | |
| | CAU05876; Cox_Guide6 of FIG. | |
| | 11 | |
| 106 | aligned sequence-LC126150 of | caactcaatgattaacaacatca |
| | FIG. 11 | |
| 107 | consensus sequence-untitled | gagacaaattacatcgactacctg |
| | consensus; aligned sequences- | |
| | AF465516; Echo_guide1 and | |
| | X89538_Echo7_3D of FIG. 12 | |
| 108 | aligned sequences- | gagacaaattacattgactacttg |
| | X89554_Echo27_3D and | |
| | X89548_Echo19_3D of FIG. 12 | |
| 109 | aligned sequence- | gagacaaactacatcgactaccta |
| | X89541_Echo12_3D of FIG. 12 | |
| 110 | consensus sequence-untitled | tcccaccacctgtacagagacaa |
| | consensus; aligned sequences- | |
| | AF465516; Echo_guide2 and | |
| | X89538_Echo7_3D of FIG. 12 | |
| 111 | aligned sequence- | tctcaccatotgtacagagataa |
| | X89554_Echo27_3D of FIG. 12 | |
| 112 | aligned sequence- | tcccaccacctgtacagagataa |
| | X89548_Echo19_3D of FIG. 12 | |
| 113 | aligned sequence- | tctcaccacctgtacagagacaa |
| | X89541_Echo12_3D of FIG. 12 | |
| 114 | consensus sequence-untitled | ttcaggatgattgcatatggtga |
| | consensus; aligned sequences- | |
| | AF465516; Echo_guide3 and | |
| | X89538 Echo7_3D of FIG. 12 | |
| 115 | aligned sequence- | ttcaggatgatagcatatggtga |
| | X89554_Echo27_3D of FIG. 12 | |
| 116 | aligned sequence- | tttaggatgatcgcatatggtga |
| | X89548_Echo19_3D of FIG. 12 | |
| 117 | aligned sequence- | tttagaatgattgcatatggtga |
| | X89541_Echo12_3D of FIG. 12 | |
| 118 | consensus sequence-untitled | ttgattatgacaccagcagataa |
| | consensus of FIG. 12 | |
| 119 | aligned sequence-AF465516; | ttggttatgacaccagcagataa |
| | Echo_guide4 and | |
| | X89538_Echo7_3D | |
| 120 | aligned sequence- | ttgattatgacaccagcagacaa |
| | X89554_Echo27_3D of FIG. 12 | |
| 121 | aligned sequence- | ttgatcatgacaccagcagataa |
| | X89548_Echo19_3D of FIG. 12 | |
| 122 | aligned sequence- | ctgattatgacaccagcagacaa |
| | X89541_Echo12_3D of FIG. 12 | |

All sequences are provided in 5′-3′, unless otherwise denoted.

SEQ ID NO: 37: pZac2.1-CMV-CasRx-3×HA-7508 bp ds-DNA circular
Sequence annotation:

### Features: Location/Qualifiers

primer: complement (121 . . . 144)/label=“2225”/note=“sequence: GGGAGAGGCGGTTTGCGTA TTGGG” (SEQ ID NO: 38)
repeat_region: 364 . . . 493/label=“ITR”
primer: 534 . . . 557/label=“EK18011”/note=“sequence: gccatgctctaggaagatcgggac” (SEQ ID NO: 39)
primer: 534 . . . 593/label=“EK18001”/note=“sequence: gccatgctctaggaagatcgggacattgattattgactagtta ttaatagtaatcaatta” (SEQ ID NO: 40)
enhancer: 555 . . . 934/label=“CMV enhancer”
CDS: 555 . . . 934/label=“Translation 555-934”
primer: 622 . . . 645/label=“1075”/note=“sequence: CGGAGTTCCGCGTTACATAACTTAC” (SEQ ID NO: 41)
primer: 722 . . . 741/label=“1324”/note=“sequence: ACGCCAATAGGGACTTTCCA” (SEQ ID NO: 42)
primer: 811 . . . 830/label=“2394”/note=“sequence: AAGTACGCCCCCTATTGACG” (SEQ ID NO: 43)
promoter: 936 . . . 1134/label=“CMV promoter”
primer: complement (1052 . . . 1077)/label=“EK18015”/note=“sequence: gagttgttacgacattttgg aaagtc” (SEQ ID NO: 44)
primer: complement (1111 . . . 1134)/label=“GG001”/note=“sequence: ATGGTCTCCatctgcttatatag acctcccaccgt” (SEQ ID NO: 45)
primer: complement (1111 . . . 1134)/label=“SV036” tgtggacttcacgcccatggtggcctgc ttatatagacctcccaccgt” (SEQ ID NO: 46)/note= “sequence:
misc_feature: 1135 . . . 1140/label=“Kozak”
misc_feature: 1141 . . . 4017/label=“CasRx”
CDS: 1141 . . . 4017/label=“Translation 1141-4017”
primer: 4018 . . . 4044/label=“GG004”/note=“sequence: ATGGTCTCGgatacccatacgatgttccaga ttacgct” (SEQ ID NO: 47)
CDS: 4018 . . . 4098/label=“3×HA”
primer: complement (4076 . . . 4100)/label=“GG005”/note=“sequence: TAGGTCTCAcattaggcatagt cggggacatcatatg” (SEQ ID NO: 48)
primer: 4102 . . . 4140/label=“GG006” ATGGTCTCCaatggctaataaaggaaatttattttca ttgcaatagtgtg” (SEQ ID NO: 49)/note=“sequence:
polyA_signal 4102 . . . 4488/label=“rabbit b-globin polyA”
primer: complement (4462 . . . 4488)/label=“GG007”/note=“sequence: ATGGTCTCActcgatctccata agagaagagggacagct” (SEQ ID NO: 50)
primer: 4489 . . . 4510/label=“U6_fwd_seqchk”/note=“sequence: gagggcctatttcccatgattc” (SEQ ID NO: 51)
primer: 4489 . . . 4514/label=“GG008”/note=“sequence: ATGGTCTCCcgagggcctatttcccatgatt ccttc” (SEQ ID NO: 52)
promoter: 4489 . . . 4729/label=“U6 promoter”
primer: complement (4490 . . . 4511)/label=“13 EF1a_R (out)”/note=“sequence: ggaatcatgggaaata ggccct” (SEQ ID NO: 53)
primer_bind: 4660 . . . 4679/label=“LKO.1 5”
primer: complement (4713 . . . 4738)/label=“736”/note=“sequence: CGGTGTTTCGTCCTTTCCA CAAGATA” (SEQ ID NO: 54)
primer: 4743 . . . 4759/label=“EK_GS_F1”/note=“sequence: cctaccaactggtcggg” (SEQ ID NO: 55)
primer: 4743 . . . 4764/label=“EK18100”/note=“sequence: cctaccaactggtcggggtttg” (SEQ ID NO: 56)
primer: complement (4791 . . . 4817)/label=“EK18101”/note=“sequence: GGAGAAAATACCGCA TCAGAATTCAAA” (SEQ ID NO: 57)
primer: complement (4796 . . . 4817)/label=“EK_GS_R1”/note=“sequence: ggagaaaataccgcatcag aat” (SEQ ID NO: 58)
primer: complement (4843 . . . 4895)/label=“EK18008”/note=“sequence: gctacttatctacgtagccatgcg aattactatggttgctttgacgtatgcgg” (SEQ ID NO: 59)
primer: complement (4866 . . . 4895)/label-“EK18012”/note=“sequence: gctacttatctacgtagccatg cgaattac” (SEQ ID NO: 60)
misc_feature: join (4868 . . . 7508, 1 . . . 554)/label=“pZac2.1 BB”
misc_feature: 4920 . . . 5060/label=“ITR”
primer: complement (5206 . . . 5225)/label=“2452”/note=“sequence: TTCAGGCTGCGCAACTGT TG” (SEQ ID NO: 61)
primer: 5824 . . . 5849/label=“1625”/note=“sequence: ATGAGTATTCAACATTTCCGTGTCGC” (SEQ ID NO: 62)
primer: complement (7226 . . . 7246)/label=“2372”/note=“sequence: GCTCACGCTGTAGGT ATCTCA” (SEQ ID NO: 63)

### Full Sequence:

tcctgcgttatcccctgattctgtggataaccgtattaccgcctttgagtgagctgataccgctcgccgcagccgaacgaccgagcgca
gcgagtcagtgagcgaggaagcggaagagcgcccaatacgcaaaccgcctctccccgcgcgttggccgattcattaatgcagctg
gcacgacaggtttcccgactggaaagcgggcagtgagcgcaacgcaattaatgtgagttagctcactcattaggcaccccaggcttt
acactttatgcttccggctcgtatgttgtgtggaattgtgagcggataacaatttcacacaggaaacagctatgaccatgattacgccag
atttaattaaggctgcgcgctcgctcgctcactgaggccgcccgggcaaagcccgggcgtcgggcgacctttggtcgcccggcctca
gtgagcgagcgagcgcgcagagagggagtggccaactccatcactaggggttccttgtagttaatgattaacccgccatgctacttat
ctacgtagccatgctctaggaagatcgggacattgattattgactagttattaatagtaatcaattacggggtcattagttcatagcccatat
atggagttccgcgttacataacttacggtaaatggcccgcctggctgaccgcccaacgacccccgcccattgacgtcaataatgacgt
atgttcccatagtaacgccaatagggactttccattgacgtcaatgggtggagtatttacggtaaactgcccacttggcagtacatcaagt
gtatcatatgccaagtacgccccctattgacgtcaatgacggtaaatggcccgcctggcattatgcccagtacatgaccttatgggacttt
cctacttggcagtacatctacgtattagtcatcgctattaccatggtgatgcggttttggcagtacatcaatgggcgtggatagcggtttga
ctcacggggatttccaagtctccaccccattgacgtcaatgggagtttgttttggcaccaaaatcaacgggactttccaaaatgtcgtaa
caactccgccccattgacgcaaatgggcggtaggcgtgtacggtgggaggtctatataagcaggccaccatgggcgtgaagtccac
actcgtgtccggctccaaagtgtacatgacaaccttcgccgaaggcagcgacgccaggctggaaaagatcgtggagggcgacag
catcaggagcgtgaatgagggcgaggccttcagcgctgaaatggccgataaaaacgccggctataagatcggcaacgccaaattc
agccatcctaagggctacgccgtggtggctaacaaccctctgtatacaggacccgtccagcaggatatgctcggcctgaaggaaact
ctggaaaagaggtacttcggcgagagcgctgatggcaatgacaatatttgtatccaggtgatccataacatcctggacattgaaaaaa
tcctcgccgaatacattaccaacgccgcctacgccgtcaacaatatctccggcctggataaggacattattggattcggcaagttctcc
acagtgtatacctacgacgaattcaaagaccccgagcaccatagggccgctttcaacaataacgataagctcatcaacgccatcaa
ggcccagtatgacgagttcgacaacttcctcgataaccccagactcggctatttcggccaggcctttttcagcaaggagggcagaaatt
acatcatcaattacggcaacgaatgctatgacattctggccctcctgagcggactgaggcactgggtggtccataacaacgaagaag
agtccaggatctccaggacctggctctacaacctcgataagaacctcgacaacgaatacatctccaccctcaactacctctacgaca
ggatcaccaatgagctgaccaactccttctccaagaactccgccgccaacgtgaactatattgccgaaactctgggaatcaaccctgc
cgaattcgccgaacaatatttcagattcagcattatgaaagagcagaaaaacctcggattcaatatcaccaagctcagggaagtgat
gctggacaggaaggatatgtccgagatcaggaaaaatcataaggtgttcgactccatcaggaccaaggtctacaccatgatggactt
tgtgatttataggtattacatcgaagaggatgccaaggtggctgccgccaataagtccctccccgataatgagaagtccctgagcgag
aaggatatctttgtgattaacctgaggggctccttcaacgacgaccagaaggatgccctctactacgatgaagctaatagaatttggag
aaagctcgaaaatatcatgcacaacatcaaggaatttaggggaaacaagacaagagagtataagaagaaggacgcccctagac
tgcccagaatcctgcccgctggccgtgatgtttccgccttcagcaaactcatgtatgccctgaccatgttcctggatggcaaggagatca
acgacctcctgaccaccctgattaataaattcgataacatccagagcttcctgaaggtgatgcctctcatcggagtcaacgctaagttcg
tggaggaatacgcctttttcaaagactccgccaagatcgccgatgagctgaggctgatcaagtccttcgctagaatgggagaacctatt
gccgatgccaggagggccatgtatatcgacgccatccgtattttaggaaccaacctgtcctatgatgagctcaaggccctcgccgaca
ccttttccctggacgagaacggaaacaagctcaagaaaggcaagcacggcatgagaaatttcattattaataacgtgatcagcaata
aaaggttccactacctgatcagatacggtgatcctgcccacctccatgagatcgccaaaaacgaggccgtggtgaagttcgtgctcgg
caggatcgctgacatccagaaaaaacagggccagaacggcaagaaccagatcgacaggtactacgaaacttgtatcggaaagg
ataagggcaagagcgtgagcgaaaaggtggacgctctcacaaagatcatcaccggaatgaactacgaccaattcgacaagaaa
aggagcgtcattgaggacaccggcagggaaaacgccgagagggagaagtttaaaaagatcatcagcctgtacctcaccgtgatct
accacatcctcaagaatattgtcaatatcaacgccaggtacgtcatcggattccattgcgtcgagcgtgatgctcaactgtacaaggag
aaaggctacgacatcaatctcaagaaactggaagagaagggattcagctccgtcaccaagctctgcgctggcattgatgaaactgc
ccccgataagagaaaggacgtggaaaaggagatggctgaaagagccaaggagagcattgacagcctcgagagcgccaaccc
caagctgtatgccaattacatcaaatacagcgacgagaagaaagccgaggagttcaccaggcagattaacagggagaaggcca
aaaccgccctgaacgcctacctgaggaacaccaagtggaatgtgatcatcagggaggacctcctgagaattgacaacaagacatg
taccctgttcagaaacaaggccgtccacctggaagtggccaggtatgtccacgcctatatcaacgacattgccgaggtcaattcctact
tccaactgtaccattacatcatgcagagaattatcatgaatgagaggtacgagaaaagcagcggaaaggtgtccgagtacttcgacg
ctgtgaatgacgagaagaagtacaacgataggctcctgaaactgctgtgtgtgcctttcggctactgtatccccaggtttaagaacctga
gcatcgaggccctgttcgataggaacgaggccgccaagttcgacaaggagaaaaagaaggtgtccggcaattccggatccggata
cccatacgatgttccagattacgcttatccctacgacgtgcctgattatgcatacccatatgatgtccccgactatgcctaatggctaataa
aggaaatttattttcattgcaatagtgtgttggaattttttgtgtctctcactcggaaggacatatgggagggcaaatcatttaaaacatcag
aatgagtatttggtttagagtttggcaacatatgcccatatgctggctgccatgaacaaaggttggctataaagaggtcatcagtatatgaaaca
gccccctgctgtccattccttattccatagaaaagccttgacttgaggttagattttttttatattttgttttgtgttatttttttctttaacatccc
taaaattttccttacatgttttactagccagatttttcctcctctcctgactactcccagtcatagctgtccctcttctcttatggagatcgagggc
ctatttcccatgattccttcatatttgcatatacgatacaaggctgttagagagataattggaattaatttgactgtaaacacaaagatatta
gtacaaaatacgtgacgtagaaagtaataatttcttgggtagtttgcagttttaaaattatgttttaaaatggactatcatatgcttaccgtaa
cttgaaagtatttcgatttcttggctttatatatcttgtggaaaggacgaaacaccgaacccctaccaactggtcggggtttgaaacgggt
cttcgagaagaccttttttttgaattctgatgcggtattttctccttacgcatctgtgcggtatttcacaccgcatacgtcaaagcaaccatagt
aattcgcatggctacgtagataagtagcatggcgggttaatcattaactacaAGGAACCCCTAGTGATGGAGTTGGC
CACTCCCTCTCTGCGCGCTCGCTCGCTCACTGAGGCCGGGCGACCAAAGGTCGCCCGAC
GCCCGGGCTTTGCCCGGGGGGCCTCAGTGAGCGAGCGAGCGCGCAGCCTAATTAAGGcct
taattaacctaattcactggccgtcgttttacaacgtcgtgactgggaaaaccctggcgttacccaacttaatcgccttgcagcacatccc
cctttcgccagctggcgtaatagcgaagaggcccgcaccgatcgcccttcccaacagttgcgcagcctgaatggcgaatgggacgc
gccctgtagcggcgcattaagcgcggcgggtgtggtggttacgcgcagcgtgaccgctacacttgccagcgccctagegcccgctcc
tttcgctttcttcccttcctttctcgccacgttcgccggctttccccgtcaagctctaaatcgggggctccctttagggttccgatttagtgcttta
cggcacctogaccccaaaaaacttgattagggtgatggttcacgtagtgggccatcgccctgatagacggtttttcgccctttgacgttg
gagtccacgttctttaatagtggactcttgttccaaactggaacaacactcaaccctatctcggtctattcttttgatttataagggattttgcc
gatttcggcctattggttaaaaaatgagctgatttaacaaaaatttaacgcgaattttaacaaaatattaacgtttataatttcaggtggcat
ctttcggggaaatgtgcgcggaacccctatttgtttatttttctaaatacattcaaatatgtatccgctcatgagacaataaccctgataaat
gcttcaataatattgaaaaaggaagagtatgagtattcaacatttccgtgtcgcccttattcccttttttgcggcattttgccttcctgtttttgctc
acccagaaacgctggtgaaagtaaaagatgctgaagatcagttgggtgcacgagtgggttacatcgaactggatctcaatagtggta
agatccttgagagttttcgccccgaagaacgttttccaatgatgagcacttttaaagttctgctatgtggcgcggtattatcccgtattgacg
ccgggcaagagcaactcggtcgccgcatacactattctcagaatgacttggttgagtactcaccagtcacagaaaagcatcttacgg
atggcatgacagtaagagaattatgcagtgctgccataaccatgagtgataacactgcggccaacttacttctgacaacgatcggag
gaccgaaggagctaaccgcttttttgcacaacatgggggatcatgtaactcgccttgatcgttgggaaccggagctgaatgaagccat
accaaacgacgagcgtgacaccacgatgcctgtagtaatggtaacaacgttgcgcaaactattaactggcgaactacttactctagct
tcccggcaacaattaatagactggatggaggcggataaagttgcaggaccacttctgcgctcggcccttccggctggctggtttattgct
gataaatctggagccggtgagcgtgggtctcgcggtatcattgcagcactggggccagatggtaagccctcccgtatcgtagttatcta
cacgacggggagtcaggcaactatggatgaacgaaatagacagatcgctgagataggtgcctcactgattaagcattggtaactgtc
agaccaagtttactcatatatactttagattgatttaaaacttcatttttaatttaaaaggatctaggtgaagatcctttttgataatctcatgac
caaaatcccttaacgtgagttttcgttccactgagcgtcagaccccgtagaaaagatcaaaggatcttcttgagatcctttttttctgcgcgt
aatctgctgcttgcaaacaaaaaaaccaccgctaccagcggtggtttgtttgccggatcaagagctaccaactctttttccgaaggtaa
ctggcttcagcagagcgcagataccaaatactgtccttctagtgtagccgtagttaggccaccacttcaagaactctgtagcaccgcct
acatacctcgctctgctaatcctgttaccagtggctgctgccagtggcgataagtcgtgtcttaccgggttggactcaagacgatagttac
cggataaggcgcagcggtcgggctgaacggggggttcgtgcacacagcccagcttggagcgaacgacctacaccgaactgagat
acctacagcgtgagctatgagaaagcgccacgcttcccgaagggagaaaggcggacaggtatccggtaagcggcagggtcgga
acaggagagcgcacgagggagcttccagggggaaacgcctggtatctttatagtcctgtcgggtttcgccacctctgacttgagcgtc
gatttttgtgatgctcgtcaggggggcggagcctatggaaaaacgccagcaacgcggcctttttacggttcctggccttttgctgcggtttt
gctcacatgttctt

1. A molecular system comprising (a) an RNA-guided RNA-targeting effector protein, and (b) one or more guide RNA molecule (gRNA); wherein each of the one or more guide RNAs comprise a guide sequence that is at least 70%, at least 80%, at least 90%, at least 95%, or 100% identical to one of the sequences set forth in SEQ ID NO: 1 to 6; and/or a molecular system comprising (a) an RNA-guided RNA-targeting effector protein, and (b) a collection of at least four different guide RNA molecules (gRNAs); wherein at least four of the gRNAs in said collection comprise a guide sequence that is at least 70%, at least 80%, at least 90%, at least 95%, or 100% identical to one of the sequences set forth in SEQ ID NO: 1 to 6.

2.-53. (canceled)

54. The molecular system of claim 1, wherein at least four of the gRNAs in said collection comprise a guide sequence that is at least 70%, at least 80%, at least 90%, at least 95%, or 100% identical to one of the sequences set forth in SEQ ID NO: 1 to 4; and/or wherein the collection comprises at least six different gRNAs, wherein six of the gRNAs in said collection comprise a guide sequence that is at least 70%, at least 80%, at least 90%, at least 95%, or 100% identical to one of the sequences set forth in SEQ ID NO: 1 to 6.

55. The molecular system of claim 1, wherein the RNA-guided RNA-targeting effector protein is an RNA-guided RNA-targeting Cas protein, or a modified variant thereof.

56. The molecular system of claim 55, wherein the Cas protein is selected from the group consisting of: a Cas13a, a Cas13b, a Cas13c, and a Cas13d.

57. The molecular system of claim 56, wherein the Cas 13d is the Cas13d orthologue of Ruminococcus flavefaciens (CasRx).

58. The molecular system of claim 57, wherein the Cas protein is a CasRx; and wherein the collection comprises six different guide RNA molecules (gRNAs), wherein each of the gRNAs comprises a guide sequence that is set forth in SEQ ID NO: 1 to 6.

59. A method of treating, preventing, suppressing, and/or alleviating a disease associated with or caused by infection, propagation, and/or replication of an RNA virus in a subject, comprising administering to a subject in need thereof a molecular system comprising the molecular system of claim 1.

60. A method of treating, preventing, suppressing, and/or alleviating a disease associated with or caused by infection, propagation and/or replication of an RNA virus in a subject, comprising administering to a subject in need thereof a molecular system comprising (a) an RNA-guided RNA-targeting effector protein and/or a polynucleic acid encoding said effector protein, and (b) one or more gRNAs and/or one or more polynucleic acids encoding said one or more gRNAs; wherein each of the one or more gRNAs comprises a guide sequence that is at least 70%, at least 80%, at least 90%, at least 95% or 100% identical to one of the sequences set forth in SEQ ID NO: 1-6; and/or a method of treating, preventing, suppressing, and/or alleviating a disease associated with or caused by infection, propagation and/or replication of an RNA virus in a subject, comprising administering to a subject in need thereof a molecular system comprising (a) an RNA-guided RNA-targeting effector protein and/or a polynucleic acid encoding said effector protein, and (b) a collection of at least four different gRNAs and/or one or more polynucleic acids encoding said collection of gRNAs; wherein at least four of the gRNAs in said collection comprise a guide sequence that is at least 70%, at least 80%, at least 90%, at least 95% or 100% identical to one of the sequences set forth in SEQ ID NO: 1-6.

61. The method of claim 60, wherein at least four of the gRNAs in said collection comprise a guide sequence that is at least 70%, at least 80%, at least 90% at least 95%, or 100% identical to one of the sequences set forth in SEQ ID NO: 1-4.

62. The method of claim 60, wherein (b) or (b1) comprises at least six different gRNAs, wherein six of the gRNAs in said collection comprise a guide sequence that is at least 70%, at least 80%, at least 90%, at least 95%, or 100% identical to one of the sequences set forth in SEQ ID NO: 1-6.

63. The method according to claim 60, wherein the polynucleic acid encoding said effector protein and/or the one or more polynucleic acids encoding said gRNAs are comprised in one or more vectors; and/or wherein the vector is a viral vector; and/or wherein the viral vector is an adenoviral vector, an Adeno-associated virus (AAV) vector, a lentiviral vector, or a retroviral vector; and/or wherein the viral vector is an AAV vector; and/or wherein the AAV vector is selected from the group consisting of: AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV9, AAV10, AAV11, AAV12, AAV13, rh10, AAVDJ, AAV-PHP.S, AAV-PHP.B, AAV-PHP.eB, and Anc80; and/or wherein the AAV vector is an AAV2, or AAVDJ, or AAV1 vector.

64. The method according to claim 60, wherein the RNA-guided RNA-targeting effector protein is an RNA-guided RNA-targeting Cas protein, or a modified variant thereof; and/or wherein the Cas protein is selected from the group consisting of: a Cas13a, a Cas13b, a Cas13c, and a Cas13d; and/or wherein the Cas 13d is the Cas13d orthologue of Ruminococcus flavefaciens (CasRx).

65. The method according to claim 60, wherein the disease is caused by an RNA virus and/or wherein the RNA virus is an Enterovirus.

66. The method according to claim 60, wherein the disease is selected from the group consisting of: polio; mild respiratory illness (the common cold); hand, foot, and mouth disease (HFMD); acute hemorrhagic conjunctivitis; aseptic meningitis; myocarditis; severe neonatal sepsis-like disease; acute flaccid paralysis; acute flaccid myelitis; Bornholm disease; epidemic pleurodynia; Herpangina; chronic fatigue syndrome; and Diabetes mellitus type 1.

67. The method according to claim 66 wherein the disease is HFMD caused by an RNA virus selected from the group consisting of Enterovirus, Coxsackie virus, and Parechovirus; and/or wherein: i) the Enterovirus is Enterovirus 71; ii) the Coxsackie virus is selected from the group consisting of CAV16 and CAV6; and iii) the Parechovirus is selected from the group consisting of Parechovirus A, Parechovirus B, Parechovirus C, Parechovirus D, Parechovirus E, and Parechovirus F.

68. A polynucleotide encoding the molecular system of claim 1.

69. A vector encoding one or more gRNAs, wherein the one or more gRNAs encoded by said vector comprise a guide sequence that is at least 70%, at least 80%, at least 90%, at least 95%, or 100% identical to one of the sequences set forth in SEQ ID NO: 1-6.

70. The vector of claim 69, wherein the vector encodes at least four different gRNAs, wherein each of the gRNAs comprises a guide sequence that is set forth in SEQ ID NO: 1-6.

71. The vector of claim 70, wherein the vector encodes six different gRNAs, wherein each of the gRNAs comprises a guide sequence that is set forth in SEQ ID NO: 1-6.

72. A vector encoding the molecular system of claim 1.

73. The vector of claim 69, wherein the vector is an adenoviral vector, an Adeno-associated virus (AAV) vector, a lentiviral vector, or a retroviral vector.

74. The vector of claim 73, wherein the vector is an AAV vector.

75. The vector of claim 74, wherein the vector is selected from the group consisting of: AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV9, AAV10, AAV11, AAV12, AAV13, rh10, AAVDJ, AAV-PHP.S, AAV-PHP.B, AAV-PHP.eB, and Anc80.

76. The vector of claim 75, wherein the AAV vector is an AAV2, AAVDJ, or AAV1 vector.

77. A composition comprising a polynucleotide of claim 68.

78. The composition of claim 77, further comprising an RNA-guided RNA-targeting effector protein.

79. The composition of claim 78, wherein the RNA-guided RNA-targeting effector protein is a Cas protein selected from the group consisting of: a Cas13a, a Cas13b, a Cas13c, and a Cas13d.

80. The composition of claim 79, wherein the Cas 13d is the Cas13d orthologue of Ruminococcus flavefaciens (CasRx).

81. A method of treating, preventing, suppressing, and/or alleviating a disease associated with or caused by infection, propagation and/or replication of an RNA virus in a subject, comprising administering to a subject in need thereof a molecular system comprising the composition of claim 77.

82. A guide RNA molecule (gRNA), comprising a guide sequence that is at least 70%, at least 80%, at least 90%, at least 95%, or 100% identical to any one of the sequences set forth in SEQ ID NO: 1-6.

83. A guide RNA molecule comprising a guide sequence as set forth in any one of SEQ ID NO: 1-6.

84. A method of treating, preventing, suppressing, and/or alleviating a disease associated with or caused by infection, propagation and/or replication of an RNA virus in a subject, comprising administering to a subject in need thereof a composition comprising the vector of claim 69.

85. A composition comprising the vector of claim 69.
